(19) United States
(12) Patent Application Publication
(10) Pub. No.: US 2026/0199466 A1
(43) Pub. Date: Jul. 16, 2026

(54) BLTP3A-SILENCED ANTIGEN-SPECIFIC ENGINEERED T CELLS

(71) Applicant: RUTGERS, THE STATE UNIVERSITY OF NEW JERSEY, New Brunswick, NJ (US)

(72) Inventors: Kyle Kristopher PAYNE, Atlantic Highlands, NJ (US); Fnu Rinkee KUMARI, Edison, NJ (US)

(21) Appl. No.: 19/445,787

(22) Filed: Jan. 12, 2026

Related U.S. Application Data
(60) Provisional application No. 63/744,662, filed on Jan. 13, 2025.

(51) Int. Cl. A61K 40/11; A61K 40/32; A61K 40/42; A61K 45/06; A61P 35/00; C07K 14/705; C07K 14/725; C12N 15/86

(52) U.S. Cl. CPC A61K 40/11; A61K 40/32; A61K 40/421; A61K 45/06; A61P 35/00; C07K 14/7051; C07K 14/70517; C12N 15/86; C12N 2750/14143

(57) ABSTRACT

Described herein are pharmaceutical compositions comprising antigen-specific engineered T cells comprising chimeric antigen receptor (CAR)-T cells, T cell receptor (TCR)-T cells, or tumor-infiltrating lymphocytes (TILs) having attenuated or terminated BLTP3A expression. In some embodiments, the antigen-specific engineered T cells have increased anti-cancer effector activity relative to antigen-specific engineered T cells having normal BLTP3A expression. Also described herein are methods of treating cancer in a subject comprising administering to the subject a therapeutically effective amount of the pharmaceutical compositions described herein.

### CROSS-REFERENCE TO RELATED APPLICATIONS

This application claims priority to U.S. Provisional Patent Application No. 63/744,662, filed on Jan. 13, 2025, which is incorporated by reference herein in its entirety.

### REFERENCE TO SEQUENCE LISTING

This application was filed with a Sequence Listing XML in ST.26 XML format accordance with 37 C.F.R. § 1.831. The Sequence Listing XML file submitted in the USPTO Patent Center, “210953-0021-US02_sequence_listing_xml_9 Jan. 2026.xml,” was created on Jan. 9, 2026, contains 130 sequences, has a file size of 156 kilobytes (159,895 bytes), and is incorporated by reference in its entirety into the specification.

### BACKGROUND

Despite recent advances in immunotherapeutic approaches for treating cancer, such treatment modalities rarely induce objective responses in cancer patients. These disappointing results are likely due to the lack of a clear understanding of the many immune suppressive mechanisms functioning within the tumor-microenvironment (TME). Recently, TME-driven endoplasmic stress response signaling has been shown to promote mitochondrial dysfunction in tumor-reactive T cells, leading to their impaired antitumor efficacy against cancer, including invasive epithelial ovarian cancer (EOC). However, the current understanding of TME-driven cellular stress responses which promote T cell dysfunction remains in its infancy.

What is needed are improved immunotherapeutic compositions and methods for treating cancer.

### SUMMARY

One embodiment described herein is a pharmaceutical composition comprising antigen-specific engineered T cells comprising chimeric antigen receptor (CAR)-T cells, T cell receptor (TCR)-T cells, or tumor-infiltrating lymphocytes (TILs) having attenuated or terminated BLTP3A expression. In one aspect, BLTP3A expression is terminated in the antigen-specific engineered T cells. In another aspect, the antigen-specific engineered T cells express a nucleic acid molecule specific to BLTP3A that maintains attenuated or terminated BLTP3A expression. In another aspect, the nucleic acid molecule is a CRISPR-Cas9 construct, a shRNA construct, a siRNA construct, a miRNA construct, or combinations thereof. In another aspect, the nucleic acid molecule is comprised in a viral vector. In another aspect, the viral vector comprises a lentiviral vector, a retroviral vector, an adenoviral vector, or an adeno-associated virus (AAV) vector. In another aspect, the antigen-specific engineered T cells having attenuated or terminated BLTP3A expression have increased anti-cancer effector activity relative to antigen-specific engineered T cells having normal BLTP3A expression. In another aspect, the pharmaceutical composition further comprises one or more pharmaceutically acceptable buffers, salts, carriers, or diluents.

Another embodiment described herein is a kit comprising: any of the pharmaceutical compositions described herein; optionally, injection or infusion materials or devices; and optionally, one or more of packaging, a label, or instructions for use.

Another embodiment described herein is a method of treating cancer in a subject, the method comprising administering to the subject a therapeutically effective amount of a pharmaceutical composition comprising antigen-specific engineered T cells comprising chimeric antigen receptor (CAR)-T cells, T cell receptor (TCR)-T cells, or tumor-infiltrating lymphocytes (TILs) having attenuated or terminated BLTP3A expression. In one aspect, the cancer is a solid-tumor cancer or a liquid cancer. In another aspect, the cancer is a solid-tumor cancer selected from pancreatic cancer, breast cancer, lung cancer, brain cancer, neck cancer, ovarian cancer, cervical cancer, prostate cancer, kidney cancer, liver cancer, colorectal cancer, stomach cancer, esophageal cancer, and skin cancer. In another aspect, the cancer is ovarian cancer. In another aspect, the pharmaceutical composition is administered to the subject by intravenous injection or infusion. In another aspect, the pharmaceutical composition delays malignant progression of the cancer in the subject. In another aspect, the pharmaceutical composition is cytotoxic to the cancer in the subject. In another aspect, the method further comprises administering to the subject a therapeutically effective amount of one or more additional anti-cancer drugs. In another aspect, the one or more additional anti-cancer drugs comprise a PD-1 inhibitor, a PD-L1 inhibitor, a CTLA-4 inhibitor, a TIM-3 inhibitor, a LAG-3 inhibitor, a CD137 agonist, or combinations thereof.

Another embodiment described herein is a method of producing antigen-specific engineered T cells comprising chimeric antigen receptor (CAR)-T cells, T cell receptor (TCR)-T cells, or tumor-infiltrating lymphocytes (TILs) having attenuated or terminated BLTP3A expression, the method comprising: treating the antigen-specific engineered T cells with a nucleic acid molecule specific to BLTP3A for a sufficient period of time to attenuate or terminate BLTP3A expression in the antigen-specific engineered T cells. In one aspect, treating the antigen-specific engineered T cells with a nucleic acid molecule comprises transfecting the T cells with a CRISPR-Cas9 construct specific to BLTP3A, transducing the T cells with a shRNA construct specific to BLTP3A, transducing the T cells with a siRNA construct specific to BLTP3A, transducing the T cells with a miRNA construct specific to BLTP3A, or combinations thereof. In another aspect, the nucleic acid molecule is comprised in a viral vector. In another aspect, the viral vector comprises a lentiviral vector, a retroviral vector, an adenoviral vector, or an adeno-associated virus (AAV) vector. In another aspect, the nucleic acid molecule is stably expressed in the antigen-specific engineered T cells to maintain attenuated or terminated BLTP3A expression. In another aspect, the method terminates BLTP3A expression in the antigen-specific engineered T cells. In another aspect, the method further comprises expanding the antigen-specific engineered T cells.

Another embodiment described herein is a method of increasing anti-cancer effector activity of antigen-specific engineered T cells comprising chimeric antigen receptor (CAR)-T cells, T cell receptor (TCR)-T cells, or tumor-infiltrating lymphocytes (TILs), the method comprising attenuating or terminating BLTP3A expression in the antigen-specific engineered T cells. In one aspect, attenuating or terminating BLTP3A expression in the antigen-specific engineered T cells comprises treating the antigen-specific engineered T cells with a nucleic acid molecule specific to BLTP3A for a sufficient period of time to attenuate or terminate BLTP3A expression in the antigen-specific engineered T cells. In another aspect, treating the antigen-specific engineered T cells with a nucleic acid molecule comprises transfecting the T cells with a CRISPR-Cas9 construct specific to BLTP3A, transducing the T cells with a shRNA construct specific to BLTP3A, transducing the T cells with a siRNA construct specific to BLTP3A, transducing the T cells with a miRNA construct specific to BLTP3A, or combinations thereof.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-C show diagrams illustrating the methods that were used to develop and study a conditional knockout mouse, where Bltp3a (Uhrf1bp1) was specifically ablated in post-thymic T cells (CD4^{Cre} Bltp3a^{f/f}). FIG. 1A shows a diagram illustrating how CD4^{Cre} Bltp3a^{f/f} mice were developed. FIG. 1B shows a diagram illustrating how BLTP3A td-Tomato mice were developed.

FIG. 1C shows a diagram illustrating that UPK10 cells, which are a mouse ovarian cancer cell line, were injected into mice having post-thymic T cells with wild-type Bltp3a expression (Bltp3a^{WT}) or no Bltp3a expression (Bltp3a^{KO}), and survival and T-cell effector functions were studied.

FIG. 2A-C show that BLTP3A^{M1098T} is associated with better survival outcome in high-grade serous ovarian cancer (HGSOC). FIG. 2A shows TCGA datasets were utilized to compare the survival of ovarian cancer patients with polymorphic BLTP3A^{M1098T} (n=76) to those with the ancestral BLTP3A allele (n=327). FIG. 2B shows multiplex immunofluorescence images of Grzb⁺ and CD3⁺ T cells among total T cells. FIG. 2C shows quantification of the multiplex immunofluorescence images shown in FIG. 2B.

FIG. 3A-C show that BLTP3A is aberrantly expressed by epithelial ovarian cancer (EOC)-infiltrating T cells in the tumor microenvironment. FIG. 3A shows PCR quantification (ΔΔCₜ) of BLTP3A transcripts relative to β-actin in CD3⁺ T cells isolated from peripheral blood and matched tumor tissue (TIL) from 3 HGSOC specimens. FIG. 3B shows FACs detection of td-Tomato-tagged Bltp3a in tumor-infiltrating CD3⁺ T cells isolated from spleen and tumor of td-Tomato-reported mice challenged with BPPNM ovarian cancer tumor cells (3×10⁶, i.p.) 35 days post tumor challenge. FIG. 3C shows FACs detection of td-Tomato-tagged BLTP3A in antigen-experienced tumor-infiltrating CD8⁺ T cells from td-Tomato mice.

FIG. 4A-E show that BLPT3A^{KO} mice demonstrate superior survival and enhanced T cell immune activity in the tumor microenvironment upon tumor challenge. FIG. 4A shows the percent survival for CD4^{Cre} Bltp3a^{f/f} (BLTP3A^{KO}) or Bltp3a^{f/f} (BLTP3A^{WT}) mice littermate controls that were challenged with BPPNM tumor cells (3×10⁶, i.p.). FIG. 4B shows the frequency of total CD3⁺ TILs from BPPNM tumors. FIG. 4C shows the frequencies of IFNγ (red) and IFNγ/Granzyme B (blue) in CD8⁺ T cells immunopurified at day 21 from parallel cohorts of mice from FIG. 4A upon restimulation with tumor antigens in vitro for 16 hr. FIG. 4D shows the frequencies of CellTrace Violet (CTV) proliferation marker and CD44 in the CD8⁺ T cells. FIG. 4E shows RNA-seq pathway analysis from BLTP3A^{KO} and BLTP3A^{WT} CD8⁺ T cells activated with α-(CD3/CD28) for 6 hr and followed by treatment with 20% ascites for 18 hr.

FIG. 5A-D show that tumor microenvironment-derived stress impairs TCR complex surface retention and TCR triggering. FIG. 5A shows Amnis ImageStream data of surface CD3 expression. FIG. 5B shows immunopurified splenic CD8⁺ T cells isolated from BLPT3A^{KO} or

BLPT3A^{WT} CD3/CD28-stimulated mice with or without 20% EOC-derived ascites for 24 hr. FIG. 5C shows TCRβ expression in tumor infiltrating T cells. FIG. 5D shows FACS quantification of pCD3ζ^{pY142} in immunopurified BLTP3A^{WT} and BLPT3A^{KO} splenic CD8⁺ T cells cultured with 20% ascites for 24 hr, followed by TCR crosslinking with plate-bound α-CD3 (3 μg/mL) plus α-CD28 (5 μg/mL) for 2 min.

FIG. 6A-C show that BLTP3A associates with retromer complex and machinery of cellular trafficking. FIG. 6A shows LC-MS/MS intensity of absolute protein quantification (iBAQ) fold-increase from DSP-crosslinked BLTP3A-FLAG immunoprecipitation (IP). FIG. 6B-C show splenic BLPT3A^{KO} or BLTP3A^{WT} CD8⁺ T cells were activated in the presence of 20% ascites for 24 hr. FIG. 6B shows cells were stained with 20 μg/mL TCRβ (green), incubated to allow endocytosis, and immediately fixed and stained for LAMP1 (clone #D2D11), or RAB4A (1:50). FIG. 6C shows fold recycling of TCRβ by flow cytometry.

FIG. 7A-C show that BLTP3A-ablated CD8⁺ T cells delay malignant progression in a model of adoptive cellular therapy and may be more responsive to immune checkpoint blockade. FIG. 7A shows a diagram illustrating how the experiments were conducted. FIG. 7B shows percent survival of syngeneic mice challenged with UPK10 tumor cells (5×10⁶, i.p.) that were treated (1×10⁶, i.v.) with ex vivo tumor-antigen primed splenic BLTP3A^{KO} T cells, BLTP3A^{WT} T cells, or vehicle (PBS) control 7 days after tumor challenge (n=5/group). FIG. 7C shows PD-1 surface expression on Bltp3a^{WT} and Bltp3a^{KO} CD8⁺ T cells isolated from tumor beds upon euthanasia.

FIG. 8 shows diagrams illustrating hypothesized molecular mechanisms for the effects of BLTP3A expression on T cell function in tumor microenvironments.

FIG. 9 shows a western blot image of BLTP3A knockdown using two different BLTP3A shRNA hairpin constructs with a doxycycline-inducible promoter.

FIG. 10 shows an exemplary BLTP3A-specific artificial microRNA (amiR-BLTP3A) MSGV1 vector design. The top image shows the principal design of BLTP3A amiRs, containing 5′-regions, loop regions, and 3′-regions, with the stem sequence being derived from short hairpin RNA (shRNA). an exemplary sequence is shown in SEQ ID NO: 129; see Table 2). A design schematic of an exemplary MSGV1 vector construct is shown on the bottom with the location of the insertion sites for the amiR sequences and the TCR/CAR. LTR, long terminal repeat; SD, splice donor; psi, extended packaging sequences; SA, splice acceptor. Exemplary miRNAs include miR-142, miR-146b, miR-150, miR-155, miR-16, and miR-223. Specific sequences are shown in Table 2.

FIG. 11 shows an immunoblot illustrating suppressed BLTP3A levels after expression of shRNA TRCN0000422652 (SEQ ID NO: 129) in Jurkat cells. BLTP3A levels were assessed after 72 hr.

### DETAILED DESCRIPTION

Described herein are pharmaceutical compositions comprising antigen-specific engineered T cells comprising chimeric antigen receptor (CAR)-T cells, T cell receptor (TCR)-T cells, or tumor-infiltrating lymphocytes (TILs) having attenuated or terminated BLTP3A expression. These tumor-antigen-specific T cells are engineered by silencing the expression of the gene BLTP3A using, for example, CRISPR/Cas9-mediated ablation or artificial shRNA/miRNA-mediated knockdown methods, to drive elevated T cell anti-cancer effector activity and control malignant progression upon therapeutic administration to cancer subjects.

BLTP3A is an uncharacterized regulator of T cell activity in cancer. Existing approaches to engineer T cells to arm their effectiveness against cancer include expressing a cell surface receptor (e.g., CAR or TCR) to redirect their activity against cancer. However, these T cells remain sensitive to T cell-intrinsic suppressive programs which dampen their ability to eliminate cancer cells in situ. The compositions and methods described herein combine T cell engineering approaches with the targeting of BLTP3A expression, which was found to mediate a T cell-intrinsic immunosuppressive program which can greatly enhance the efficacy of cellular therapeutics for cancer. These findings can be applied to various cellular therapies for the treatment of cancers (both solid and liquid) in the context of antigen-specific engineered T cells (e.g., CAR-T cells, TCR-T cells, or TILs).

In some embodiments described herein, antigen-specific engineered T cells are transduced or transfected with shRNA, miRNA, and/or CRISPR-Cas9 constructs to silence BLTP3A expression. The therapeutic efficacy of the BLTP3A-depleted engineered T cells can then be evaluated against cancer. These engineered T cells having attenuated or terminated BLTP3A expression may exhibit superior anti-tumor activity relative to antigen-specific engineered T cells having normal BLTP3A expression. The anti-cancer efficacy of the BLTP3A-silenced or ablated engineered T cells described herein may also be synergized with immune checkpoint blockade therapies, such as PD-1 inhibitors.

### Definitions

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “gene,” “nucleic acid,” “nucleotide,” “polynucleotide,” “oligonucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein. Nucleic acids may be single stranded or double stranded or may contain portions of both double stranded and single stranded sequence. The nucleic acid may be DNA, both genomic and cDNA, RNA (e.g., mRNA, siRNA, shRNA, miRNA), or a hybrid, where the nucleic acid may contain combinations of deoxyribo- and ribo-nucleotides, and combinations of bases including uracil, adenine, thymine, cytosine, guanine, inosine, xanthine, hypoxanthine, isocytosine, and isoguanine. Nucleic acids may be obtained by chemical synthesis methods or by recombinant methods.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claim. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points, or as described above in the definition of “about.”

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers a temperature of about 15-30° C., including all integers and endpoints within the specified range. In another aspect, “room temperature” refers a temperature of about 15-30° C.; about 20-30° C.; about 22-30° C.; about 25-30° C.; about 27-30° C.; about 15-22° C.; about 15-25° C.; about 15-27° C.; about 20-22° C.; about 20-25° C.; about 20-27° C.; about 22-25° C.; about 22-27° C.; about 25-27° C.; about 15° C.±10%; about 20° C.±10%; about 22° C.±10%; about 25° C.±10%; about 27° C.±10%; ~20° C., ~22° C., ~25° C., or ~27° C., at standard atmospheric pressure.

As used herein, “substantial identity” of polynucleotide sequences means that a polynucleotide comprises a sequence that has at least 25% sequence identity compared to a reference sequence as determined using programs known in the art (e.g., Basic Local Alignment Search Tool (BLAST)). In preferred embodiments, percent identity can be any integer from 25% to 100%. More preferred embodiments include polynucleotide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. These values can be appropriately adjusted to determine corresponding identity of proteins encoded by two nucleotide sequences by taking into account codon degeneracy, amino acid similarity, reading frame positioning, and the like. Accordingly, polynucleotides of the present disclosure encoding a protein or polypeptide of the present disclosure include nucleic acid sequences that have substantial identity to the nucleic acid sequences that encode the proteins or polypeptides of the present disclosure. Polynucleotides encoding a polypeptide comprising an amino acid sequence that has at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference polypeptide sequence are also preferred.

As used herein, “substantial identity” of amino acid sequences (and of polypeptides having these amino acid sequences) means that an amino acid sequence comprises a sequence that has at least 25% sequence identity compared to a reference sequence as determined using programs known in the art (e.g., BLAST). In preferred embodiments, percent identity can be any integer from 25% to 100%. More preferred embodiments include amino acid or polypeptide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. Polypeptides that are “substantially identical” share amino acid sequences except that residue positions which are not identical may differ by one or more conservative amino acid changes, as described above. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acid substitution groups include valine-leucine-isoleucine, phenylalanine-tyrosine, lysine-arginine, alanine-valine, aspartic acid-glutamic acid, and asparagine-glutamine. Accordingly, polypeptides or proteins, encoded by the polynucleotides of the present disclosure, include amino acid sequences that have substantial identity to the amino acid sequences of the reference polypeptide sequences.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

In some embodiments disclosed herein, a subject may be administered a single dose of the disclosed cells or pharmaceutical compositions. In other embodiments, a subject may be administered a plurality of doses over a period of time. For example, in various embodiments, a cell or pharmaceutical composition as described herein may be administered to a subject once a day (SID/QD), twice a day (BID), three times a day (TID), four times a day (QID), or more, so as to administer a therapeutically effective amount of the cell or pharmaceutical composition to the subject, where the therapeutically effective amount is any one or more of the doses described herein. In some embodiments, a cell or pharmaceutical composition as described herein is administered to a subject 1-3 times per day, 1-7 times per week, 1-9 times per month, 1-12 times per year, or more. In other embodiments, a cell or pharmaceutical composition as described herein is administered for about 1-10 days, 10-20 days, 20-30 days, 30-40 days, 40-50 days, 50-60 days, 60-70 days, 70-80 days, 80-90 days, 90-100 days, 1-6 months, 6-12 months, 1-5 years, or more. In various embodiments, a cell or pharmaceutical composition as described herein is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, 900-1000 mg/kg, or a combination thereof. The actual dosing regimen can depend upon many factors, including but not limited to, the judgment of a trained physician, the overall condition of the subject, and the specific disease or condition of the subject. The actual dosage can also depend on the determined experimental effectiveness of the specific cell or pharmaceutical composition that is administered. For example, the dosage may be determined based on in vitro responsiveness of relevant cultured cells, or in vivo responses observed in appropriate animal models or human studies.

As used herein, the term “endogenous” refers to any material from or produced inside an organism, cell, tissue, or system.

As used herein, the term “exogenous” refers to any material introduced from or produced outside an organism, cell, tissue, or system.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “administering” refers to the placement of a cell or a composition as disclosed herein into a subject by a method or route which results in at least partial localization of the cell or composition at a desired site. “Route of administration” may refer to any administration pathway known in the art, including but not limited to oral, intravenous (IV), topical, aerosol, nasal, via inhalation, anal, intra-anal, peri-anal, transmucosal, transdermal, parenteral, enteral, or local. “Parenteral” refers to a route of administration that is generally associated with injection, including intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous (IV), intraarterial, subarachnoid, subcapsular, subcutaneous, transmucosal, or transtracheal. Via the parenteral route, the cell or composition may be in the form of solutions or suspensions for IV infusion or IV injection, or as lyophilized powders. Via the enteral route, the cell or composition can be in the form of capsules, gel capsules, tablets, sugar-coated tablets, syrups, suspensions, solutions, powders, granules, emulsions, microspheres or nanospheres or lipid vesicles or polymer vesicles allowing controlled release. Via the topical route, the cell or composition can be in the form of aerosol, lotion, cream, gel, ointment, suspensions, solutions, or emulsions. In accordance with the present disclosure, “administering” can be self-administering. For example, it is considered “administering” when a subject injects or consumes a cell or composition as disclosed herein.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifested.

As used herein, the term “cytotoxic” or “cytotoxicity” refers to killing or damaging cells.

As used herein, “sample” or “target sample” refers to any sample in which the presence and/or level of a target analyte or target biomarker is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological or bodily fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

As used herein, “target analyte,” “target biomarker,” “target antigen,” or “target cell” refers to a substance that is associated with a biological state or a biological process, such as a disease state or a diagnostic or prognostic indicator of a disease or disorder (e.g., an indicator identifying the likelihood of the existence or later development of a disease or disorder). The presence or absence of a biomarker, or the increase or decrease in the concentration or expression of a biomarker, can be associated with and/or be indicative of a particular state or process. Biomarkers can include, but are not limited to, cells or cellular components (e.g., a viral cell, a bacterial cell, a fungal cell, a cancer cell, a tumor cell, etc.), small molecules, lipids, carbohydrates, nucleic acids, peptides, proteins, enzymes, antigens, and antibodies. A biomarker can be derived from an infectious agent, such as a bacterium, fungus, or virus, or can be an endogenous molecule that is found in greater or lesser abundance in a subject suffering from a disease or disorder as compared to a normal healthy individual (e.g., an increase or decrease in expression of a gene or gene product).

As used herein, the term “lentivirus” or “lentiviral” refers to a genus of the Retroviridae family. Lentiviruses are unique among the retroviruses in being able to infect non-dividing cells; they can deliver a significant amount of genetic information into the DNA of the host cell, so they are one of the most efficient methods of a nucleic acid delivery vector. HIV, SIV, and FIV are all examples of lentiviruses. Vectors derived from lentiviruses (i.e., lentiviral vectors) offer the means to achieve efficient transduction of exogenous nucleic acid molecules in vivo. In certain non-limiting exemplary embodiments of the present disclosure, a lentiviral vector may be used to modify an antigen-specific engineered T cell to express a nucleic acid molecule that modulates the expression of BLTP3A.

As used herein, “expression” means the process by which a gene ultimately results in a protein. Expression includes, but is not limited to, transcription, post-transcriptional modification (e.g., splicing, polyadenylation, addition of 5′-cap), and translation.

As used herein, the term “cancer” refers to a disease characterized by the rapid and uncontrolled growth of aberrant cells. Cancer cells can spread locally or through the bloodstream and lymphatic system to other parts of the body. Examples of various cancers include, but are not limited to, breast cancer, prostate cancer, ovarian cancer, cervical cancer, skin cancer, pancreatic cancer, colorectal cancer, renal or kidney cancer, liver cancer, brain cancer, neck cancer, stomach cancer, esophageal cancer, lymphoma, blood cancer, leukemia, myeloma, lung cancer, and the like. As used herein, the term “solid-tumor cancer” refers to a malignancy where abnormal cells form one or more localized tissue masses (i.e., tumors) rather than circulating freely in the blood or bone marrow. “Solid-tumor cancer” may also refer to a “non-blood cancer,” as commonly known in the art. In certain embodiments of the present disclosure, solid-tumor cancer may comprise pancreatic cancer, breast cancer, lung cancer, brain cancer, neck cancer, ovarian cancer, cervical cancer, prostate cancer, kidney cancer, liver cancer, colorectal cancer, stomach cancer, esophageal cancer, or skin cancer. As used herein, the term “liquid cancer” refers to a malignant neoplasm that originates in the blood, bone marrow, or lymphatic system and is characterized by cancerous cells circulating within bodily fluids rather than forming a localized solid tumor mass. In certain embodiments of the present disclosure, a subject may be treated for a liquid cancer and/or a solid-tumor cancer.

As used herein, the term “chimeric antigen receptor” or “CAR” refers to an artificial cell surface receptor that is engineered to be expressed on an immune effector cell and specifically bind to an antigen. CARs may be used as a therapy with adoptive cell transfer. The structure of CAR constructs may be modulated based on the intended target antigen and the specific immune cell type comprising the CAR. Immune effector cells such as antigen-specific T cells may be removed from a patient (blood, tumor, or ascites fluid) and further modified (e.g., using a lentiviral vector expression system) so that they express CARs specific to a particular form of antigen.

As used herein, the term “attenuated” means reduced or decreased gene or protein expression. As used herein, the term “terminated” means no gene or protein expression (i.e., complete silencing or knockout of expression).

As used herein, the term “anti-cancer effector activity” refers to biological processes by which the antigen-specific engineered T cells described herein recognize, inhibit, and/or destroy cancer cells and tumor cells. As used herein, “anti-cancer effect” or “anti-tumor effect” refers to a biological effect which can be manifested by a decrease in cancer cell number or cancer cell proliferation, a decrease in tumor volume, a decrease in the number of tumor cells, a decrease in activity of pro-tumor or oncogenic molecular signaling transduction, an increase in activity of tumor suppressive molecular signaling transduction, an increase in activity of pro-immune or pro-inflammatory molecular signaling transduction, a decrease in the number of metastases, an increase in life expectancy, amelioration of various physiological symptoms associated with a cancerous condition, and the like. An “anti-cancer effect” can also be manifested by the ability of the disclosed cells and compositions to prevent the occurrence of a cancer cell or tumor in the first place. Treating cancer can be described by a number of different parameters including, but not limited to, reduction in the size of a tumor in an animal having cancer, reduction in the growth or proliferation of a tumor in an animal having cancer, delaying malignant progression, increasing tumor cell death (i.e., cytotoxicity), preventing metastasis or reducing the extent of metastasis, and/or extending the survival of an animal having cancer. In some embodiments, the disclosed cells and compositions result in an anti-cancer effect by delaying malignant progression and increasing cytotoxicity of cancer in a subject.

### Pharmaceutical Compositions

Described herein are pharmaceutical compositions comprising antigen-specific engineered T cells having attenuated or terminated BLTP3A expression. The antigen-specific engineered T cells may comprise chimeric antigen receptor (CAR)-T cells, T cell receptor (TCR)-T cells, tumor-infiltrating lymphocytes (TILs), or combinations thereof. CAR-T cells are T cells that have been genetically modified to express an artificial cell surface receptor that specifically binds to a target antigen, such as a tumor-associated antigen. TCR-T cells are T cells that have been genetically modified to express a T cell receptor specific for a particular antigen-MHC complex. TILs are T cells that have naturally infiltrated tumor tissue and may be isolated, expanded, and optionally further modified for therapeutic use. In various aspects, the pharmaceutical compositions may be useful for treating cancer in a subject.

In some embodiments, BLTP3A expression may be attenuated or terminated in the antigen-specific engineered T cells using various nucleic acid molecules. In some aspects, the nucleic acid molecule may be a CRISPR-Cas9 construct, a shRNA construct, a siRNA construct, a miRNA construct, or combinations thereof. CRISPR-Cas9 constructs may be used to permanently disrupt the BLTP3A gene, resulting in terminated BLTP3A expression. shRNA, siRNA, and miRNA constructs may be used to knockdown BLTP3A expression, resulting in attenuated BLTP3A expression. In some embodiments, the nucleic acid molecule may be comprised in a vector, such as a viral vector. In some aspects, the viral vector may comprise a lentiviral vector, a retroviral vector, an adenoviral vector, or an adeno-associated virus (AAV) vector. In some aspects, the retroviral vector may comprise a gamma-retroviral vector. Gamma-retroviral vectors are derived from murine leukemia viruses and can be used for stable gene transfer into dividing cells. In some aspects, the gamma-retroviral vector may comprise, for example, a mouse stem cell virus (MSCV)-based vector, a murine leukemia virus (MLV)-based vector, or a modified splice-gag vector. In certain nonlimiting exemplary embodiments, the gamma-retroviral vector is an MSGV1 vector. The MSGV1 vector is a modified splice-gag vector that provides efficient transduction and stable expression of transgenes in T cells. In some aspects, the MSGV1 vector may comprise one or more of a long terminal repeat (LTR), a splice donor (SD), an extended packaging sequence (psi), or a splice acceptor (SA). In some embodiments, the MSGV1 vector may be configured to express both a gene silencing construct (such as an artificial miRNA and/or shRNA targeting BLTP3A) and a therapeutic transgene (such as a CAR or TCR) from a single promoter. In some aspects, the vector may include insertion sites for artificial miRNA sequences positioned within the vector backbone to enable simultaneous expression of gene silencing constructs and antigen-targeting receptors. Gamma-retroviral vectors, including MSGV1 vectors, offer advantages for T cell engineering including high transduction efficiency, stable genomic integration, and the ability to achieve sustained transgene expression in engineered T cells. In some embodiments, the nucleic acid molecule may be stably expressed in the antigen-specific engineered T cells to maintain attenuated or terminated BLTP3A expression over time.

In some embodiments, the nucleic acid molecules may include a single promoter or may include several promoters to drive expression of the gene silencing constructs. In some aspects, a single promoter may be used to drive expression of both a gene silencing construct and a therapeutic transgene, such as a CAR or TCR. In other aspects, multiple promoters may be used to independently control expression of different components within the same vector construct. In some embodiments, the nucleic acid constructs may include artificial miRNA designs combined with shRNA stem sequences and TCR/CAR sequences that are co-expressed from a single promoter (see e.g., FIG. 10). Such artificial miRNA-shRNA hybrid constructs may provide enhanced gene silencing efficiency while simplifying vector design by utilizing a single promoter approach. In some aspects, the artificial miRNA designs may incorporate stem sequences derived from short hairpin RNA (shRNA) flanked by 5′- and 3′-sequences and loop structures that facilitate proper processing and gene silencing activity.

In some embodiments, the pharmaceutical compositions may further comprise one or more pharmaceutically acceptable excipients. In some aspects, the pharmaceutically acceptable excipients may comprise buffers, salts, carriers, diluents, or combinations thereof. In some embodiments, the pharmaceutical compositions may be formulated for various routes of administration. In some aspects, the pharmaceutical compositions may be formulated for intravenous injection or infusion. In some aspects, the pharmaceutical compositions may be formulated as solutions or suspensions suitable for parenteral administration.

In some embodiments, the antigen-specific engineered T cells having attenuated or terminated BLTP3A expression may have increased anti-cancer effector activity relative to antigen-specific engineered T cells having normal BLTP3A expression. Without being bound to a particular theory, BLTP3A may be involved in regulating T cell receptor (TCR) trafficking and recycling within tumor microenvironments, and attenuating or terminating BLTP3A expression may enhance TCR surface retention and TCR triggering.

### Methods of Treatment

Also described herein are methods of treating cancer in a subject comprising administering to the subject a therapeutically effective amount of a pharmaceutical composition comprising antigen-specific engineered T cells having attenuated or terminated BLTP3A expression. The antigen-specific engineered T cells may comprise chimeric antigen receptor (CAR)-T cells, T cell receptor (TCR)-T cells, tumor-infiltrating lymphocytes (TILs), or combinations thereof.

In some embodiments, the cancer may be a solid-tumor cancer or a liquid cancer. In some aspects, the solid-tumor cancer may be selected from one or more of pancreatic cancer, breast cancer, lung cancer, brain cancer, neck cancer, ovarian cancer, cervical cancer, prostate cancer, kidney cancer, liver cancer, colorectal cancer, stomach cancer, esophageal cancer, and skin cancer. In certain nonlimiting exemplary embodiments, the cancer is ovarian cancer. As demonstrated in the examples described herein, BLTP3A-ablated T cells showed superior anti-tumor activity in mouse models of ovarian cancer, including epithelial ovarian cancer (EOC) and high-grade serous ovarian cancer (HGSOC). Without being bound to a particular theory, BLTP3A is aberrantly expressed by EOC-infiltrating T cells in the tumor microenvironment, and attenuating or terminating BLTP3A expression may restore T cell effector function in the tumor microenvironment.

In some embodiments, the pharmaceutical composition may be administered to the subject by intravenous injection or infusion. In some aspects, the pharmaceutical composition may be administered as an adoptive cellular therapy.

In some embodiments, the subject may be a human subject. The subject may be an adult, an adolescent, a child, or an infant. In some aspects, the subject may be a non-human mammal or animal, such as a mouse, rat, rabbit, dog, cat, pig, cow, sheep, goat, horse, or non-human primate.

In some embodiments, administration of the pharmaceutical composition may delay malignant progression of the cancer in the subject. In some aspects, administration of the pharmaceutical composition may be cytotoxic to the cancer in the subject. In some aspects, administration of the pharmaceutical composition may result in improved survival outcomes for the subject.

In some embodiments, the methods may further comprise administering to the subject a therapeutically effective amount of one or more additional anti-cancer drugs. In some aspects, the one or more additional anti-cancer drugs may comprise immune checkpoint inhibitors or agonists. In some embodiments, the one or more additional anti-cancer drugs may comprise a PD-1 inhibitor, a PD-L1 inhibitor, a CTLA-4 inhibitor, a TIM-3 inhibitor, a LAG-3 inhibitor, a CD137 agonist, or combinations thereof. As demonstrated in the examples described herein, BLTP3A-ablated CD8⁺ T cells exhibited altered PD-1 surface expression compared to wild-type T cells, suggesting that BLTP3A-ablated T cells may be more responsive to immune checkpoint blockade therapies. Without being bound to a particular theory, the combination of BLTP3A-silenced antigen-specific engineered T cells with immune checkpoint inhibitors may provide synergistic anti-cancer effects.

### Kits

Also described herein are kits comprising pharmaceutical compositions comprising antigen-specific engineered T cells having attenuated or terminated BLTP3A expression. In some embodiments, the kits may further comprise injection or infusion materials or devices. In some aspects, the kits may comprise syringes, needles, catheters, infusion bags, or infusion pumps. In some embodiments, the kits may further comprise one or more of packaging, a label, or instructions for use. In some aspects, the instructions for use may provide guidance on dosing, administration routes, storage conditions, or treatment protocols.

The kits described herein may be designed for use in various clinical settings. In some embodiments, the kit may be designed for use in a hospital or infusion center, where trained healthcare professionals can administer the compositions under close supervision. In other embodiments, the kit may be designed for use in an outpatient clinic or physician's office. The kit may include all necessary components and detailed instructions to facilitate safe and effective administration in these settings. The instructions may be tailored to the level of expertise expected of the user, with more detailed guidance provided for settings where less specialized personnel may be involved in preparation or administration. The kits described herein may comprise protective packaging to ensure the integrity of the components during storage and transport. In some embodiments, the kit may comprise cushioning materials, such as foam inserts or bubble wrap, to protect glass vials or ampoules from breakage. The kit may comprise tamper-evident seals or packaging to ensure that the kit has not been opened or tampered with prior to use. Instructions included in kits may be affixed to packaging material or may be included as a package insert. While the instructions are typically written on printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### EMBODIMENTS

One embodiment described herein is a pharmaceutical composition comprising antigen-specific engineered T cells comprising chimeric antigen receptor (CAR)-T cells, T cell receptor (TCR)-T cells, or tumor-infiltrating lymphocytes (TILs) having attenuated or terminated BLTP3A expression. In one aspect, BLTP3A expression is terminated in the antigen-specific engineered T cells. In another aspect, the antigen-specific engineered T cells express a nucleic acid molecule specific to BLTP3A that maintains attenuated or terminated BLTP3A expression. In another aspect, the nucleic acid molecule is a CRISPR-Cas9 construct, a shRNA construct, a siRNA construct, a miRNA construct, or combinations thereof. In another aspect, the nucleic acid molecule is comprised in a viral vector. In another aspect, the viral vector comprises a lentiviral vector, a retroviral vector, an adenoviral vector, or an adeno-associated virus (AAV) vector. In another aspect, the antigen-specific engineered T cells having attenuated or terminated BLTP3A expression have increased anti-cancer effector activity relative to antigen-specific engineered T cells having normal BLTP3A expression. In another aspect, the pharmaceutical composition further comprises one or more pharmaceutically acceptable buffers, salts, carriers, or diluents.

Another embodiment described herein is a kit comprising: any of the pharmaceutical compositions described herein; optionally, injection or infusion materials or devices; and optionally, one or more of packaging, a label, or instructions for use.

Another embodiment described herein is a method of treating cancer in a subject, the method comprising administering to the subject a therapeutically effective amount of a pharmaceutical composition comprising antigen-specific engineered T cells comprising chimeric antigen receptor (CAR)-T cells, T cell receptor (TCR)-T cells, or tumor-infiltrating lymphocytes (TILs) having attenuated or terminated BLTP3A expression. In one aspect, the cancer is a solid-tumor cancer or a liquid cancer. In another aspect, the cancer is a solid-tumor cancer selected from pancreatic cancer, breast cancer, lung cancer, brain cancer, neck cancer, ovarian cancer, cervical cancer, prostate cancer, kidney cancer, liver cancer, colorectal cancer, stomach cancer, esophageal cancer, and skin cancer. In another aspect, the cancer is ovarian cancer. In another aspect, the pharmaceutical composition is administered to the subject by intravenous injection or infusion. In another aspect, the pharmaceutical composition delays malignant progression of the cancer in the subject. In another aspect, the pharmaceutical composition is cytotoxic to the cancer in the subject. In another aspect, the method further comprises administering to the subject a therapeutically effective amount of one or more additional anti-cancer drugs. In another aspect, the one or more additional anti-cancer drugs comprise a PD-1 inhibitor, a PD-L1 inhibitor, a CTLA-4 inhibitor, a TIM-3 inhibitor, a LAG-3 inhibitor, a CD137 agonist, or combinations thereof.

Another embodiment described herein is a method of producing antigen-specific engineered T cells comprising chimeric antigen receptor (CAR)-T cells, T cell receptor (TCR)-T cells, or tumor-infiltrating lymphocytes (TILs) having attenuated or terminated BLTP3A expression, the method comprising: treating the antigen-specific engineered T cells with a nucleic acid molecule specific to BLTP3A for a sufficient period of time to attenuate or terminate BLTP3A expression in the antigen-specific engineered T cells. In one aspect, treating the antigen-specific engineered T cells with a nucleic acid molecule comprises transfecting the T cells with a CRISPR-Cas9 construct specific to BLTP3A, transducing the T cells with a shRNA construct specific to BLTP3A, transducing the T cells with a siRNA construct specific to BLTP3A, transducing the T cells with a miRNA construct specific to BLTP3A, or combinations thereof. In another aspect, the nucleic acid molecule is comprised in a viral vector. In another aspect, the viral vector comprises a lentiviral vector, a retroviral vector, an adenoviral vector, or an adeno-associated virus (AAV) vector. In another aspect, the nucleic acid molecule is stably expressed in the antigen-specific engineered T cells to maintain attenuated or terminated BLTP3A expression. In another aspect, the method terminates BLTP3A expression in the antigen-specific engineered T cells. In another aspect, the method further comprises expanding the antigen-specific engineered T cells.

Another embodiment described herein is a method of increasing anti-cancer effector activity of antigen-specific engineered T cells comprising chimeric antigen receptor (CAR)-T cells, T cell receptor (TCR)-T cells, or tumor-infiltrating lymphocytes (TILs), the method comprising attenuating or terminating BLTP3A expression in the antigen-specific engineered T cells. In one aspect, attenuating or terminating BLTP3A expression in the antigen-specific engineered T cells comprises treating the antigen-specific engineered T cells with a nucleic acid molecule specific to BLTP3A for a sufficient period of time to attenuate or terminate BLTP3A expression in the antigen-specific engineered T cells. In another aspect, treating the antigen-specific engineered T cells with a nucleic acid molecule comprises transfecting the T cells with a CRISPR-Cas9 construct specific to BLTP3A, transducing the T cells with a shRNA construct specific to BLTP3A, transducing the T cells with a siRNA construct specific to BLTP3A, transducing the T cells with a miRNA construct specific to BLTP3A, or combinations thereof.

Another embodiment described herein is the use of any of the pharmaceutical compositions described herein as a medicament for treating cancer in a subject.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Example 1

Development of Conditional Knockout, Bone Marrow Chimeric, and td-Tomato Mice

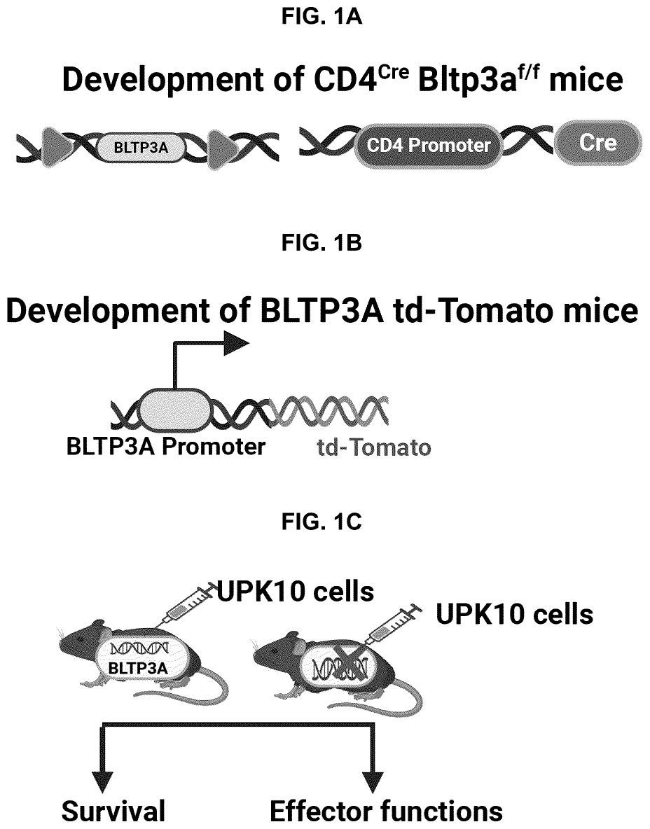

To generate Bltp3a conditional knockout (KO) mice, Bltp3a was knocked out in all mature post-thymic T cells using the Cre/lox system under the control of the T cell specific CD4 promoter (CD4^{Cre} Bltp3a^{f/f} mice) (FIG. 1A). No T cell developmental defects or pathologies were observed in these mice.

To generate Bltp3a bone marrow chimeric KO mice, CD45.2⁺ Bltp3a1^{KO} bone marrow (isolated from CD4^{Cre} Bltp3a^{f/f} mice) was admixed 1:1 with congenic CD45.1⁺ Bltp3a^{WT} bone marrow, then transplanted intravenously into congenic recipient mice that had undergone prior irradiation (2×550 rads). After 8 weeks, blood was collected retro-orbitally and the presence of CD45.1⁺ and CD45.2⁺ T cells was confirmed by flow cytometry.

To generate Bltp3a td-Tomato reporter mice, the C-terminus of the BLTP3A gene was fused to the td-Tomato reporter sequence to ensure that the td-Tomato signal directly correlated with Bltp3a protein expression (FIG. 1B).

### Survival Assay

CD4^{Cre} Bltp3a^{f/f} KO mice or Bltp3a^{f/f} wild-type (WT) littermate controls (n=5/group) were challenged with the highly aggressive murine ovarian cancer cell line UPK10 intraperitoneally (5×10⁶; i.p.) and survival and effector functions were assessed (FIG. 1C). Animals were euthanized when ascites accumulation generated a 15% gain in pre-challenge weight. In a parallel set of experiments, tumors were harvested, and tumor weights were measured.

### Flow Cytometry

For quantification of BLPT3A protein expression in different T cell subsets, td-Tomato mice were injected with BPPNM murine ovarian cancer cells (3×10⁶, i.p.). On day 25, tumors were harvested, and a single-cell suspension was prepared. Cells were first stained with Live-Dead Zombie NIR at room temperature (RT) for 10 min. After washing with PBS, cells were incubated on ice with Fc block (1 μg/1×10⁶ cells) for 5 min, followed by surface staining with the following markers for 20 min on ice: CD45 BV421, CD3 APC, CD4 PE-Cy7, CD8 PerCP5.5, CD44 Alexa Fluor 700, CD62L FITC, CD103 PE, and CD69 BV605. After staining, cells were washed with staining buffer, fixed with 1% formaldehyde at RT for 20 min, and data acquisition was done using Cytek Aurora flow cytometer.

### RNA Sequencing

To study the differential gene expression in BLTP3A^{WT} and BLTP3A^{KO} CD8⁺ T cells in the tumor microenvironment (TME), RNA sequencing was performed. CD8⁺ T cells were immunopurified from splenic cells isolated from either CD4^{Cre} Bltp3a^{f/f} KO mice or age-matched WT controls. CD8⁺ T cells were initially stimulated with Dynabeads T cell activator for 6 hr, followed by de-beading. Subsequently, the T cells were further activated for 24 hr using 20% ascitic fluid derived from ID8^{vegf/defb}-challenged mice to mimic the TME. RNA was extracted, and RNA quality was validated using a Nanodrop spectrophotometer, ensuring A₂₆₀/A₂₈₀ and A₂₆₀/A₂₃₀ ratios of 2.0.

### Blue Native PAGE and Immunoblot Analysis of TCR Nanocluster

Immunopurified CD8⁺ T cells from splenic cells isolated from Bltp3a^{KO} or age-matched Bltp3a^{WT} mice were stimulated with Dynabeads T cell activator with or without 20% EOC-derived ascites for 24 hr. Cells were lysed with a buffer containing 20 mM Bis-Tris (pH 7.0), 500 mM ε-aminocaproic acid, 20 mM NaCl, 2 mM EDTA, 10% glycerol, and 1% digitonin. TCRs were immunoprecipitated using anti-phosphotyrosine (pTyr)-conjugated Protein G beads (Sigma) and eluted with 1 M phenyl phosphate at 4° C. for 30 min. Eluted TCRs were separated with a native gel and immunoblotted with anti-CD3ζ (CST 88083).

Flow-Based Detection of pCD3ζ^{Tyr194} and pLCK^{Tyr394}

Immunopurified BLTP3A^{WT} and BLPT3A^{KO} splenic CD3⁺ T cells were cultured with 20% ascites for 24 hr, followed by TCR crosslinking with plate-bound α-CD3 (3 μg/mL, clone: 145-2C11) and α-CD28 (5 μg/mL, clone: 37.51) for 2 min at 37° C. After TCR crosslinking, the cells were immediately placed on ice and stained with Zombie NIR, CD4 BV421, CD8 FITC and pLCK^{Tyr394} PE (Clone: A18002D) or pCD3ζ^{Tyr142} PE (Clone: 3ZBR4S). Data acquisition was performed using Cytek Aurora flow cytometer.

### TCR Recycling

Immunopurified CD8⁺ T cells from splenic cells isolated from BLTP3A^{WT} and BLPT3A^{KO} mice were stimulated with Dynabeads T cell activator with or without 20% EOC-derived ascites for 24 hr. Surface TCRβ was labeled by staining cells with anti-mouse TCRβ chain antibody (20 μg/mL, clone: H57-597) for 30 min on ice and then incubated at 37° C. for 15 min for endocytosis to occur. Subsequently, the cells were stained with Zombie NIR, CD8 FITC and TCRβ-PE (clone H57-597) to measure the newly recycled surface TCRβ. The fold change of surface TCRβ was calculated relative to activated T cells exposed to ascites but without undergoing endocytosis.

### Liquid Chromatography Tandem Mass Spectrometry (LC-MS/MS) Analysis

Jurkat T cells were transduced with C-terminal FLAG-tagged lentiviral plasmid or empty plasmid. BLTP3A protein complexes were then cross-linked using DSP (Thermo Fisher) at a final concentration of 2 mM and incubated for 30 min at RT. The reaction was stopped using Tris, pH 7.5, at a final concentration of 20 mM. The cells were then washed extensively in PBS and resuspended in lysis buffer (50 mM Tris. HCl PH 8.0, 1 mM EDTA, 1% NP-40, 150 mM NaCl, and protease inhibitors), rotated at 4° C. for 30 min, and cleared by centrifugation at 14,000×g for 10 min. Cell lysates were incubated for 20 min at RT with Pierce™ Anti-FLAG Magnetic Agarose. Beads were washed in PBS with 0.02% Tween 20 three times. Proteins were eluted with LDS sample buffer supplemented with DTT by heating at 70° C. for 10 min. Eluted fractions were run on an SDS-gel for 0.5 cm, and the entire gel lanes were excised and digested with trypsin, and LC-MS/MS analysis of tryptic digests was performed. Readouts were assessed against empty plasmid.

### Confocal Microscopy

To study the colocalization of TCRβ with RAB4A⁺ recycling endosome or LAMP1⁺ lysosome, confocal microscopy was performed. Immunopurified CD8⁺ T cells from splenic cells isolated from BLTP3A^{WT} and BLPT3A^{KO} mice were stimulated with Dynabeads T cell activator with or without 20% EOC-derived ascites for 24 hr. Surface TCRβ was labeled by staining cells with anti-mouse TCRβ chain antibody (20 μg/mL, clone H57-597) on ice for 30 min. Following staining, cells were incubated at 37° C. for 15 min to allow endocytosis. Fixation was performed immediately using 2% formaldehyde for 30 min at RT. Slides were prepared using a Cytospin, followed by an additional fixation step in 2% formaldehyde for 10 min. Slides were then blocked using 5% normal serum in PBS containing 0.03% Triton X-100, and then incubated overnight at 4° C. with primary antibodies LAMP1 Rabbit mAb (clone D2D11, 1:100) or RAB4A Rabbit mAb (1:50). Cells were then stained with goat anti-armenian hamster IgG (H+L) Alexa Fluor™ 488 (Green) and Anti-rabbit IgG (H+L), F(ab′) 2 AF594 (Red) for 2 hr. Cells were then mounted using Prolong® Gold AntiFade reagent with DAPI for nuclear staining. Images were acquired without bias and were representative of approximately 30 cells per slide.

### Multiplex Immunofluorescence

A sample of 35 high-grade serous ovarian cancer (HGSOC) specimens was interrogated for the presence of BLTP3A single nucleotide polymorphism (SNP) (rs13205210) using TaqMan probes (ThermoFisher, cat. no. 4351379), where 13 specimens carrying the BLTP3A^{M1098T} allele were identified within this preliminary cohort. Utilizing formalin-fixed paraffin-embedded (FFPE) specimens, GrzB⁺ (D6E9W) CD3⁺ (2GV6) T cells among total epithelial cells (Pan-CK; AE1/AE3/PCK26), and iEP GznB⁺ CD3⁺ T cells in HGSOCs expressing ancestral BLTP3A, M1098T BLTP3A in heterozygosity (M1098THET) or homozygosity (M1098THOM) among total epithelial cells (Pan-CK; clone AE1/AE3/PCK26) were quantified. Signals for each fluorophore were calculated in Visiopharm. Multiplex Maxima was used to detect signal combinations. Total tumor sections were analyzed without bias. * p≤0.05 (n=35).

### Adoptive Cellular Therapy

Syngeneic mice that were challenged with murine UPK10 tumor cells (5×10⁶; i.p.) were treated (1×10⁶; i.v.) with ex vivo tumor-antigen primed splenic Bltp3a^{KO} CD8⁺ T cells, Bltp3a^{WT} CD8⁺ T cells, or vehicle (PBS) control 7 days after tumor challenge (n=5/group). Animals were euthanized when ascites accumulation generated a 15% gain in pre-challenge weight.

### Example 2

BLTP3A^{M1098T} is Associated with Better Survival Outcome in HGSOC

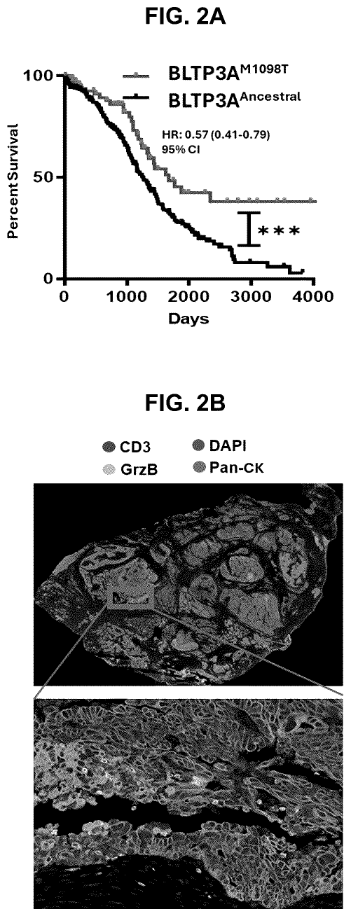

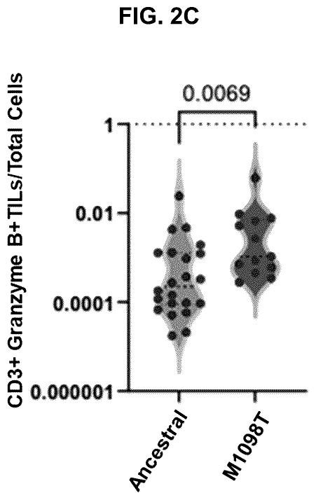

FIG. 2A-C show that BLTP3A^{M1098T} is associated with better survival outcome in high-grade serous ovarian cancer (HGSOC). FIG. 2A shows TCGA datasets were utilized to compare the survival of ovarian cancer patients with polymorphic BLTP3A^{M1098T} (n=76) to those with the ancestral BLTP3A allele (n=327). FIG. 2B shows multiplex immunofluorescence images of GrzB⁺ and CD3⁺ T cells among total T cells. FIG. 2C shows quantification of the multiplex immunofluorescence images shown in FIG. 2B.

### Example 3

### BLTP3A is Aberrantly Expressed by Epithelial Ovarian Cancer (EOC)-Infiltrating T Cells in the TME

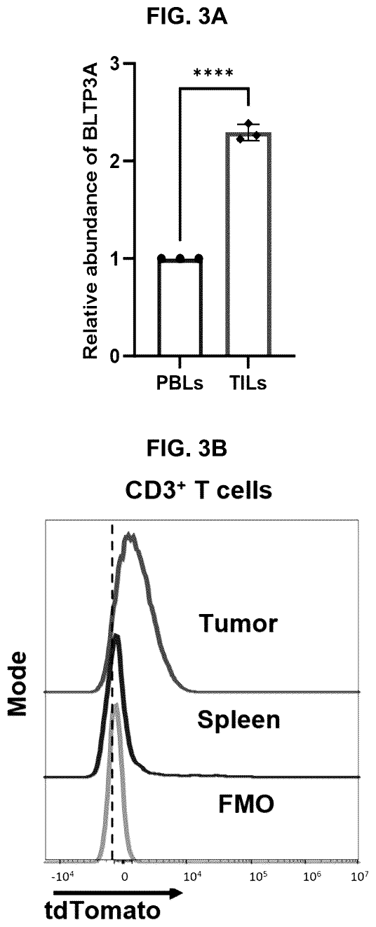

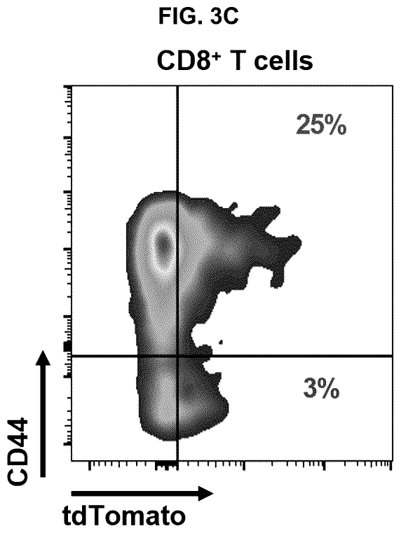

FIG. 3A-C show that BLTP3A is aberrantly expressed by epithelial ovarian cancer (EOC)-infiltrating T cells in the tumor microenvironment (TME). FIG. 3A shows PCR quantification (ΔΔCₜ) of BLTP3A transcripts relative to β-actin in CD3⁺ T cells isolated from peripheral blood and matched tumor tissue (TIL) from 3 HGSOC specimens. FIG. 3B shows FACs detection of td-Tomato-tagged Bltp3a in tumor-infiltrating CD3⁺ T cells isolated from spleen and tumor of td-Tomato-reported mice challenged with BPPNM ovarian cancer tumor cells (3×10⁶, i.p.) 35-days post tumor challenge. FIG. 3C shows FACs detection of td-Tomato-tagged BLTP3A in antigen-experienced tumor-infiltrating CD8⁺ T cells from td-Tomato mice.

### Example 4

### BLPT3A^{KO} Mice Demonstrate Superior Survival and Enhanced T Cell Immune Activity in the TME Upon Tumor Challenge

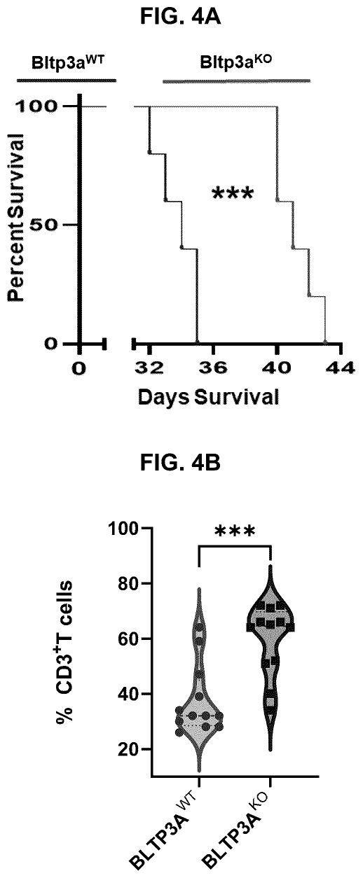

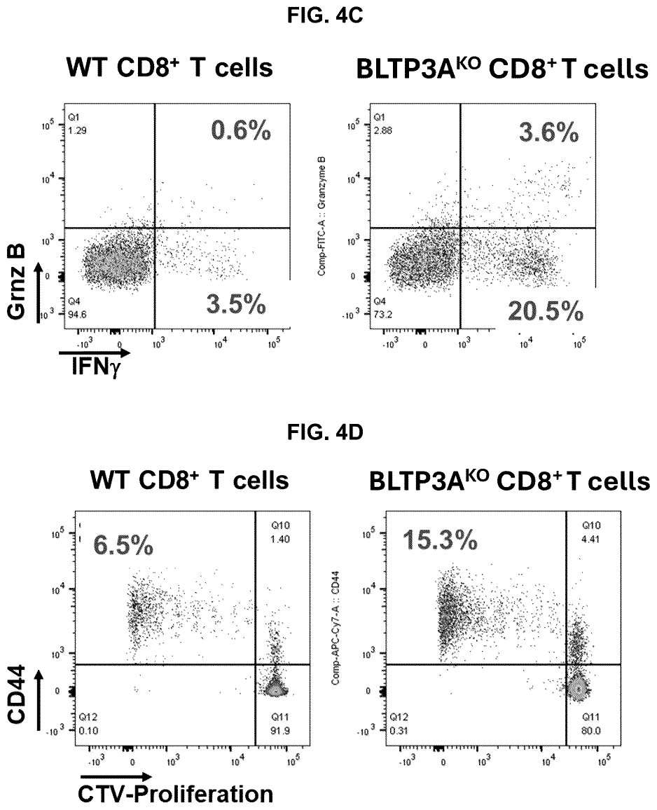

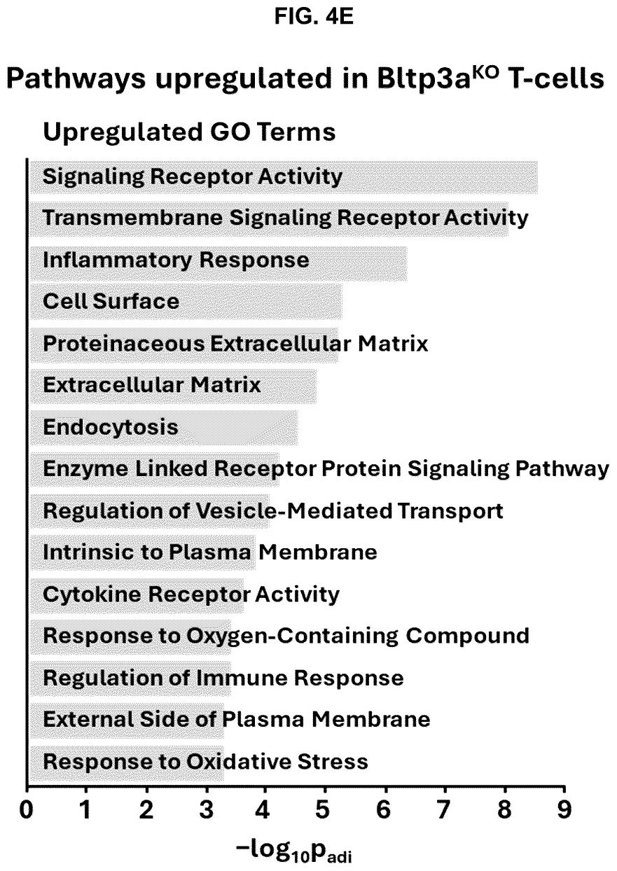

FIG. 4A-E show that BLPT3A^{KO} mice demonstrate superior survival and enhanced T cell immune activity in the TME upon tumor challenge. FIG. 4A shows the percent survival for CD4^{Cre} Bltp3a^{f/f} (BLTP3A^{KO}) or Bltp3a^{f/f} (BLTP3A^{WT}) mice littermate controls that were challenged with BPPNM tumor cells (3×10⁶, i.p.). Animals were euthanized when ascites accumulation generated a 15% gain in pre-challenge weight. FIG. 4B shows the frequency of total CD3⁺ TILs from BPPNM tumors. FIG. 4C shows the frequencies of IFNγ (red) and IFNγ/Granzyme B (blue) in CD8⁺ T cells immunopurified at day 21 from parallel cohorts of mice from FIG. 4A upon restimulation with tumor antigens in vitro for 16 hr. FIG. 4D shows the frequencies of CellTrace Violet (CTV) proliferation marker and CD44 in the CD8⁺ T cells. FIG. 4E shows RNA-seq pathway analysis from BLTP3A^{KO} and BLTP3A^{WT} CD8⁺ T cells activated with α-(CD3/CD28) for 6 hr and followed by treatment with 20% ascites for the next 18 hr.

### Example 5

### TME-Derived Stress Impairs TCR Complex Surface Retention and TCR Triggering

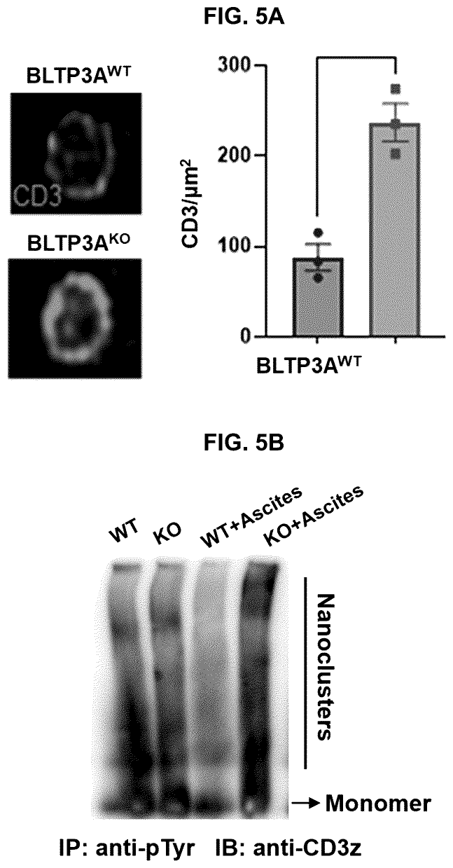

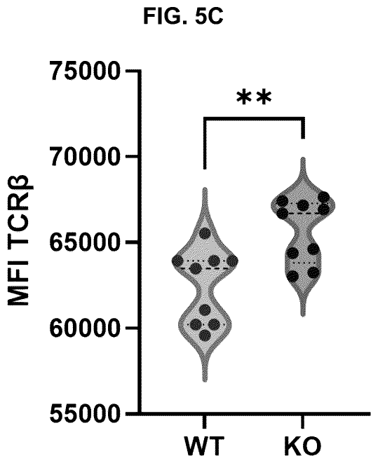

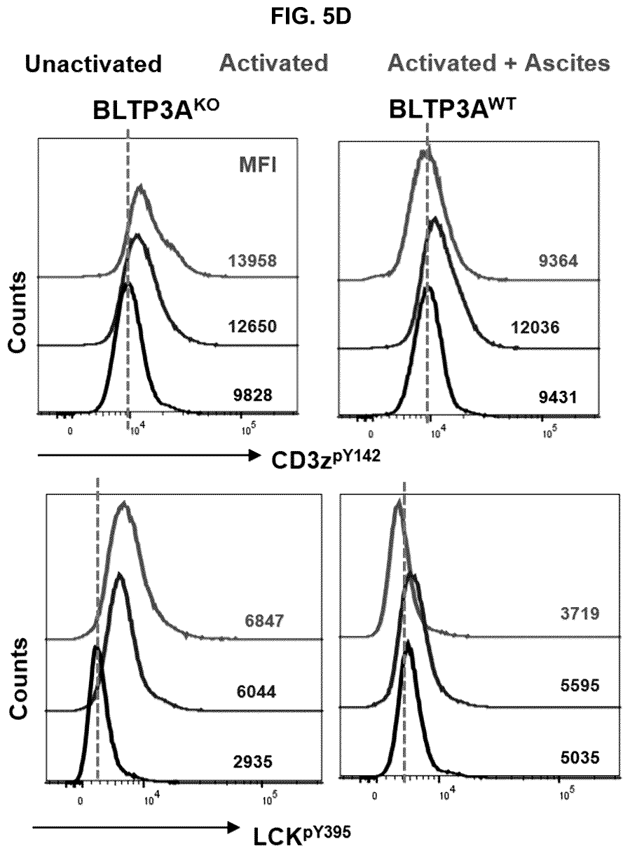

FIG. 5A-D show that TME-derived stress impairs TCR complex surface retention and TCR triggering. FIG. 5A shows Amnis ImageStream data of surface CD3 expression. FIG. 5B shows immunopurified splenic CD8⁺ T cells isolated from BLPT3A^{KO} or BLPT3A^{WT} CD3/CD28-stimulated mice with or without 20% EOC-derived ascites for 24 hr. T cells were lysed and TCRs were immunoprecipitated using anti-pTyr conjugated beads. Eluted TCRs were separated with a native gel and immunoblotted with anti-CD3ζ. FIG. 5C shows TCRβ expression in tumor infiltrating T cells. FIG. 5D shows FACS quantification of pCD3ζ^{pY142} in immunopurified BLTP3A^{WT} and BLPT3A^{KO} splenic CD8⁺ T cells cultured with 20% ascites for 24 hr, followed by TCR crosslinking with plate-bound α-CD3 (3 μg/mL) plus α-CD28 (5 μg/mL) for 2 min.

### Example 6

BLTP3A Associates with Retromer Complex and Machinery of Cellular Trafficking

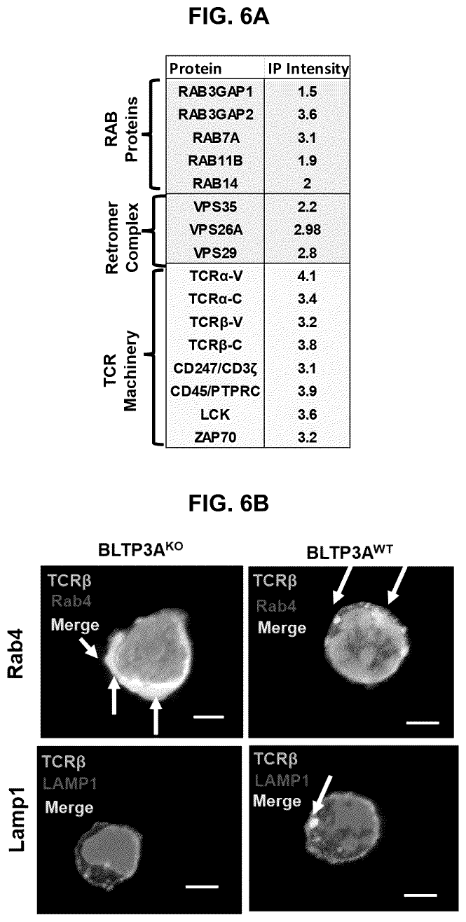

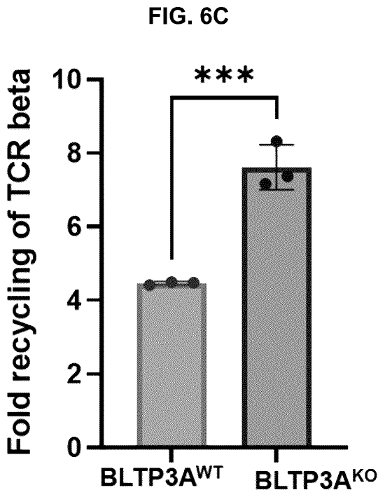

FIG. 6A-C show that BLTP3A associates with retromer complex and machinery of cellular trafficking. FIG. 6A shows LC-MS/MS intensity of absolute protein quantification (iBAQ) fold-increase from DSP-crosslinked BLTP3A-FLAG immunoprecipitation (IP). IP Intensity is the average intensity of biological triplicates with p≤0.05 over the intensity of empty vector control IP from Jurkat cells. FIG. 6B-C show splenic BLPT3A^{KO} or BLTP3A^{WT} CD8⁺ T cells were activated in the presence of 20% ascites for 24 hr. FIG. 6B shows cells were stained with 20 μg/mL TCRβ (green), incubated to allow endocytosis, and immediately fixed and stained for LAMP1 (clone #D2D11), or RAB4A (1:50). FIG. 6C shows fold recycling of TCRβ by flow cytometry.

### Example 7

### BLTP3A-ablated CD8⁺ T Cells Delay Malignant Progression in a Model of Adoptive Cellular Therapy and May Be More Responsive to Immune Checkpoint Blockade

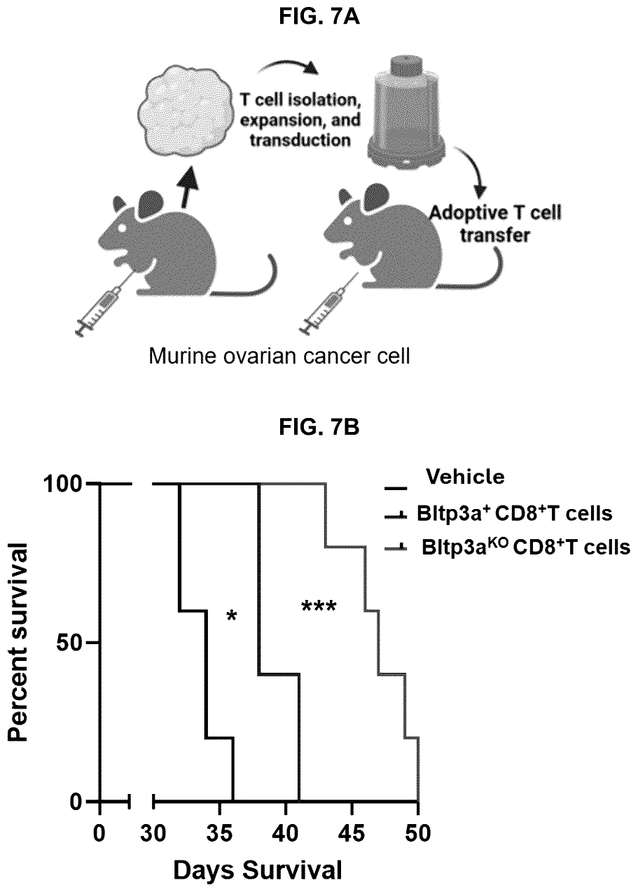

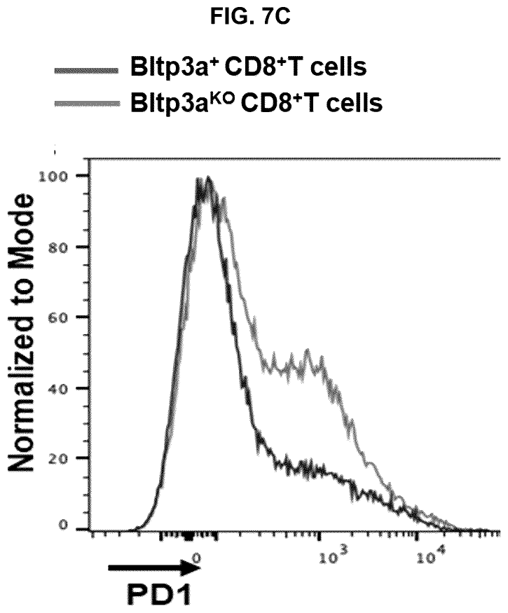

FIG. 7A-C show that BLTP3A-ablated CD8⁺ T cells delay malignant progression in a model of adoptive cellular therapy and may be more responsive to immune checkpoint blockade. FIG. 7A shows a diagram illustrating how the experiments were conducted. FIG. 7B shows percent survival of syngeneic mice challenged with UPK10 tumor cells (5×10⁶, i.p.) that were treated (1×10⁶, i.v.) with ex vivo tumor-antigen primed splenic BLTP3A^{KO} T cells, BLTP3A^{WT} T cells, or vehicle (PBS) control 7 days after tumor challenge (n=5/group). Animals were euthanized when ascites accumulation generated a 15% gain in pre-challenge weight. FIG. 7C shows PD-1 surface expression on Bltp3a^{WT} and Bltp3a^{KO} CD8⁺ T cells isolated from tumor beds upon euthanasia.

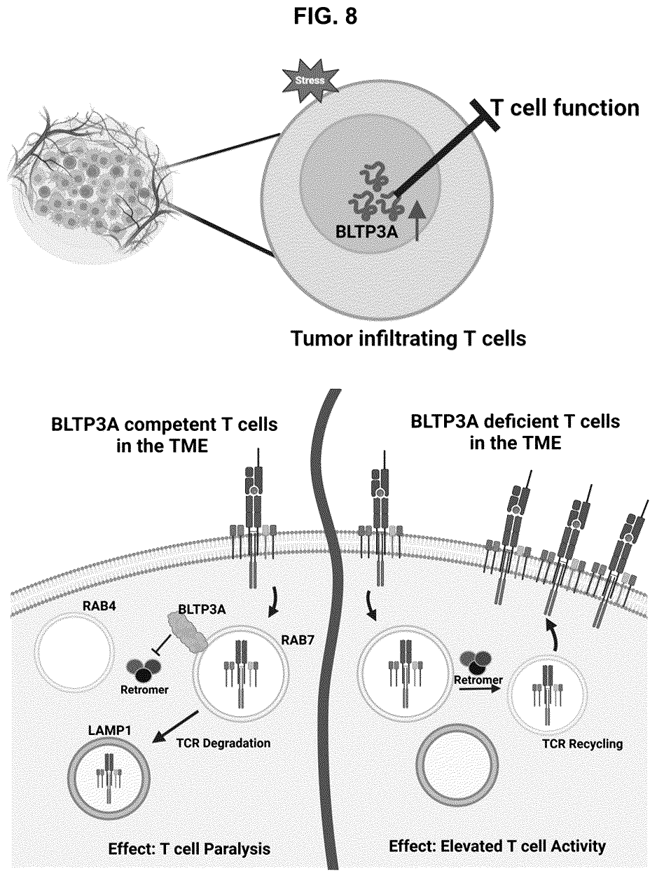

FIG. 8 shows diagrams illustrating the hypothesized molecular mechanisms for the effects of BLTP3A expression on T cell function in TMEs. In the EOC TME, aberrant BLTP3A expression is transcriptionally driven by ATF4, a known mediator of stress response signaling. It is hypothesized that this elevated BLTP3A activity drives the loss of antigen-specific T cell receptor (TCR) complexes from the cell surface to restrict TCR triggering and nanoclustering, resulting in functionally weakened TCR-peptide MHC avidities and T cell paralysis, which facilitates EOC progression. This occurs through BTLP3A-driven reprogramming of endosomal cargo sorting in tumor-specific T cells, as it was found that BLTP3A associates with RAB7⁺ endosomes and the endosomal cargo-retrieval retromer complex. Indeed, stress-driven BLTP3A outcompetes the ability of the retromer complex to bind RAB7. This results in the impairment of TCR complex retrieval from late endosomes to recycling endosomes, thereby driving the degradation of antigen reactive TCR complexes in lysosomes, which ultimately promotes EOC immune evasion (FIG. 8). Conversely, mutated BLTP3A M1098T (i.e., deficient BLTP3A) is a poor RAB7 effector which propels retromer-dependent recycling of antigen-specific TCRs back to the T cell surface where they continue to elicit protective T cell activity in EOC (FIG. 8). Collectively, this work has uncovered a previously uncharacterized immunosuppressive paradigm in EOC through BLTP3A-mediated endo/lysosomal degradation of antigen-specific TCRs.

### Example 8

Knockdown of BLTP3A Expression using shRNA Sequence Constructs Two different shRNA hairpin sequence constructs targeting BLTP3A were developed for BLTP3A silencing. The forward and reverse primer sequences used to construct each shRNA expression vector are provided in Table 1.

TABLE 1
shRNA Sequence Primers for Silencing BLTP3A

| shRNA | | SEQ |
|---|---|---|
| Name | Sequence (5′→3′) | ID NO |
| shRNA 1 F | AATTATCATGTCTTGCCTGTATAAACTCGAGT | 1 |
| | TTATACAGGCAAGACATGATTTTTTAT | |
| shRNA 1 R | TAAAAAAAATCATGTCTTGCCTGTATAAACTC | 2 |
| | GAGTTTATACAGGCAAGACATGAT | |
| shRNA 2 F | AATTACCATCAGCTGAAGTACTTAACTCGAGT | 3 |
| | TAAGTACTTCAGCTGATGGTTTTTTAT | |
| shRNA 2 R | TAAAAAAAACCATCAGCTGAAGTACTTAACTC | 4 |
| | GAGTTAAGTACTTCAGCTGATGGT | |

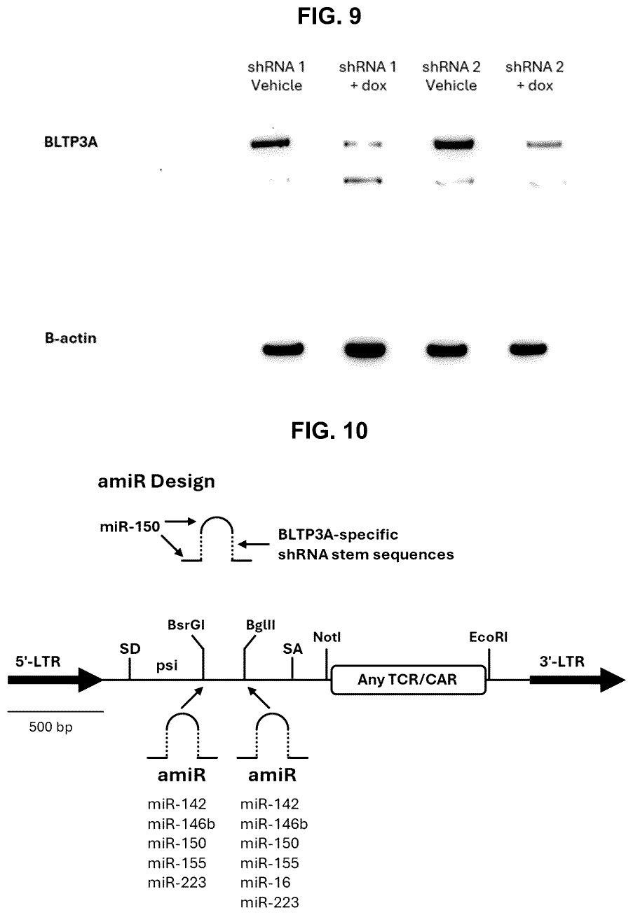

The two different shRNA constructs containing BLTP3A target sequences were cloned into the Tet-pLKO-neo lentiviral vector (Addgene; Plasmid No. 21916; SEQ ID NO: 5). Ampicillin resistant cells were sent to Azenta for sequencing. DNA from colonies containing the correct hairpin sequence was isolated and used for transfection with HEK293T cells (5×10⁶ cells per well in 6-well). Cells were transfected with 10 μg Tet-pLKO-neo, 7.5 μg packaging plasmid psPAX2, and 2.5 μg envelope plasmid pMD2.G using JetPrime DNA/siRNA transfection reagent (Polyplus). Supernatant containing lentivirus was collected at 72 hr and concentrated using LentiX Concentrator (Clontech). 0.3×10⁶ OVCAR8 cells were seeded in a 6-well plate for lentiviral transduction in DMEM/10% FBS media containing 5 μg/mL polybrene. 1 mL of lentiviral supernatant was added to each well and incubated for 24 hr before growth medium was replaced with fresh DMEM/10% FBS containing no polybrene. Cells were expanded before selection with doxycycline hyclate. 0.4×10⁶ transduced cells were seeded for treatment with growth medium containing 100 ng/ml doxycycline hyclate (diluted in tissue culture grade water) for 48 hr. Cells were then lysed in the plate with ice-cold RIPA lysis buffer (Millipore Sigma) containing 1×Halt Protease Inhibitor Cocktail (Thermo Fisher). Lysate was quantified using Pierce BCA Protein Assay Kit and 20 μg of protein was loaded onto a NuPAGE™ 10%, Bis-Tris gel (Invitrogen) for western blotting. Membranes were blocked in 5% skim milk 1×TBST and incubated with anti-UHRF1BP1 Rabbit anti-Human antibody (Bethyl Laboratories) and developed using ECL Prime Western Blotting Detection Reagents (Cytiva) to confirm BLTP3A gene silencing. FIG. 9 shows a representative western blot image demonstrating that BLTP3A expression was reduced using both shRNA constructs as compared to the vehicle controls.

### Example 9

### In Vitro Functional Efficacy of T Cells Upon BLTP3A Knockdown

The two BLTP3A shRNA hairpins of Example 8 were constitutively expressed in primary peripheral T cells using the pLKO.3G lentiviral vector backbone (Addgene; Plasmid No. 14748; SEQ ID NO: 6). Both hairpins were found to effectively suppress BLTP3A expression in the T cells.

Additional functional efficacy studies of the BLTP3A-depleted T cells are conducted.

### Example 10

BLTP3A-Specific Artificial microRNA (amiR-BLTP3A) for BLTP3A Knockdown

FIG. 10 shows a design schematic for co-expression of artificial miRNAs (amiR) targeting BLTP3A and an antigen-specific T cell receptor (TCR) or chimeric antigen receptor (CAR) in MSGV. The top image shows the principle design of BLTP3A-targeting amiRs, containing the 5′-loop, and 3′-sequences, with stem sequences being derived from short hairpin RNA (shRNA). An exemplary MSGV1 vector construct is shown on the bottom with the location of the insertion sites for the amiR sequences and the TCR/CAR. LTR, long terminal repeat; SD, splice donor; psi, extended packaging sequence; SA, splice acceptor. Exemplary miRNAs include miR-142, miR-146b, miR-150, miR-155, miR-16, and miR-223. This approach utilizes a single vector system co-expressing a BLTP3A-silencing amiR with shRNA stem sequences and a TCR or CAR all from the same promoter. Sequences for various constructs are shown in Table 2.

TABLE 2
BLTP3A Targeting amiR Sequences

| | | |
|---|---|---|
| | | SEQ |
| | | ID |
| Name | Sequences (5′→3′) | NO |

amiR Backbone Sequences

| | | |
|---|---|---|
| miR-16 5′- | cctcaaaaatacaaggatctgatcttctgaagaaaatatatttcttttt | 7 |
| Sequence | attcatagctcttatgatagcaatgtcagcagtgcct | |
| miR-142 5′- | acaaggagggctggggggctcttggagcaggagtcaggaggcctgggca | 8 |
| Sequence | gcctgaagagtacacgccgacggacagacagacagtgcagtcacc | |
| miR-146b 5′- | ttactcatcctgggaacgggagacgattcacagaagaaagcatgcaaga | 9 |
| Sequence | gcagcgtccaggctgaaagaactttggccacctggcac | |
| miR-150 5′- | ggacctgggtataaggcagggactgggcccacggggaggcagcgtcccc | 10 |
| Sequence | gaggcagcagcggcagcggcggctcctctccccatggccctg | |
| miR-155 5′- | acaaaccaggaaggggaaatctgtggtttaaattctttatgcctcatcc | 11 |
| Sequence | tctgagtgctgaaggcttgctgtaggctgtatgctg | |
| miR-2235′- | cctttctctctctttccctctagggtcacatctcccaggatgatctcac | 12 |
| Sequence | ttccccacagaagctcttggcctggcctcctgcagtgccacgctc | |
| miR-16 3′- | agtaaggttgaccatactctacagttgtgttttaatgtatattaatgtt | 13 |
| Sequence | actaatgtgttttcagttttattgatagtcttttcagtatt | |
| miR-142 3′- | tgagtgtactgtgggcttcggagatcacgccactgctgccgcccgctgc | 14 |
| Sequence | ccgccaccatcttcctcggcgctcggggacctcgtgtg | |
| miR-146b 3′- | gcccggcagtgctacaacatcaatgccaaggccgtggggcagctgatgg | 15 |
| Sequence | tttgggctcccaacttcccagccaggtgcttctgcag | |
| miR-150 3′- | ggacctggggaccccggcaccggcaggccccaaggggtgaggtgagcgg | 16 |
| Sequence | gcattgggacctcccctccctgtactcccatct | |
| miR-155 3′- | gtgtatgatgcctgttactagcattcacatggaacaaattgctgccgtg | 17 |
| Sequence | ggaggatgacaaagaagcatgagtcaccctgctgg | |
| miR-223 3′- | agtgcggcacatgcttaccagctctaggccagggcagatgggatatgac | 18 |
| Sequence | gaatggactgccagctggatacaaggatgctca | |
| miR-16 Loop | ttaagattctaaaattatct | 19 |
| miR-142 Loop | aacagcactggaggg | 20 |
| miR-146b Loop | tgagctctagcaat | 21 |
| miR-150 Loop | ctgggctcagacc | 22 |
| miR-155 Loop | tttgcctccaactga | 23 |
| miR-223 Loop | ggacactccatgtggtagag | 24 |

BLTP3A Targeting Sequences

| | | |
|---|---|---|
| 5′-Stem | ACCATCAGCTGAAGTACTTAA | 25 |
| Sequence 1 | | |
| 3′-Stem | TTAAGTACTTCAGCTGATGGT | 26 |
| Sequence 1 | | |
| miR-16 | cctcaaaaatacaaggatctgatcttctgaagaaaatatatttcttttt | 27 |
| | attcatagctcttatgatagcaatgtcagcagtgcctACCATCAGCTGA | |
| | AGTACTTAAttaagattctaaaattatctTTAAGTACTTCAGCTGATGG | |
| | Tagtaaggttgaccatactctacagttgtgttttaatgtatattaatgt | |
| | tactaatgtgttttcagttttattgatagtcttttcagtatt | |
| miR-142 | acaaggagggctggggggctcttggagcaggagtcaggaggcctgggca | 28 |
| | gcctgaagagtacacgccgacggacagacagacagtgcagtcacc | |
| | ACCATCAGCTGAAGTACTTAAaacagcactggagggTTAAGTACTTCAG | |
| | CTGATGGTtgagtgtactgtgggcttcggagatcacgccactgctgccg | |
| | cccgctgcccgccaccatcttcctcggcgctcggggacctcgtgtg | |
| miR-146b | ttactcatcctgggaacgggagacgattcacagaagaaagcatgcaaga | 29 |
| | gcagcgtccaggctgaaagaactttggccacctggcacACCATCAGCTG | |
| | AAGTACTTAAtgagctctagcaatTTAAGTACTTCAGCTGATGGTgccc | |
| | ggcagtgctacaacatcaatgccaaggccgtggggcagctgatggtttg | |
| | ggctcccaacttcccagccaggtgcttctgcag | |
| miR-150 | ggacctgggtataaggcagggactgggcccacggggaggcagcgtcccc | 30 |
| | gaggcagcagcggcagcggcggctcctctccccatggccctgACCATCA | |
| | GCTGAAGTACTTAActgggctcagaccTTAAGTACTTCAGCTGATGGTg | |
| | gacctggggaccccggcaccggcaggccccaaggggtgaggtgagcggg | |
| | cattgggacctcccctccctgtactcccatct | |
| miR-155 | acaaaccaggaaggggaaatctgtggtttaaattctttatgcctcatcc | 31 |
| | tctgagtgctgaaggcttgctgtaggctgtatgctgACCATCAGCTGAA | |
| | GTACTTAAtttgcctccaactgaTTAAGTACTTCAGCTGATGGTgtgta | |
| | tgatgcctgttactagcattcacatggaacaaattgctgccgtgggagg | |
| | atgacaaagaagcatgagtcaccctgctgg | |
| miR-223 | cctttctctctctttccctctagggtcacatctcccaggatgatctcac | 32 |
| | ttccccacagaagctcttggcctggcctcctgcagtgccacgctcACCA | |
| | TCAGCTGAAGTACTTAAggacactccatgtggtagagTTAAGTACTTCA | |
| | GCTGATGGTagtgcggcacatgcttaccagctctaggccagggcagatg | |
| | ggatatgacgaatggactgccagctggatacaaggatgctca | |
| 5′-Stem | ATCATGTCTTGCCTGTATAAA | 33 |
| Sequence 2 | | |
| 3′-Stem | TTTATACAGGCAAGACATGAT | 34 |
| Sequence 2 | | |
| miR-16 | cctcaaaaatacaaggatctgatcttctgaagaaaatatatttcttttt | 35 |
| | attcatagctcttatgatagcaatgtcagcagtgcctATCATGTCTTGC | |
| | CTGTATAAAttaagattctaaaattatctTTTATACAGGCAAGACATGA | |
| | Tagtaaggttgaccatactctacagttgtgttttaatgtatattaatgt | |
| | tactaatgtgttttcagttttattgatagtcttttcagtatt | |
| miR-142 | acaaggagggctggggggctcttggagcaggagtcaggaggcctgggca | 36 |
| | gcctgaagagtacacgccgacggacagacagacagtgcagtcaccATCA | |
| | TGTCTTGCCTGTATAAAaacagcactggagggTTTATACAGGCAAGACA | |
| | TGATtgagtgtactgtgggcttcggagatcacgccactgctgccgcccg | |
| | ctgcccgccaccatcttcctcggcgctcggggacctcgtgtg | |
| miR-146b | ttactcatcctgggaacgggagacgattcacagaagaaagcatgcaaga | 37 |
| | gcagcgtccaggctgaaagaactttggccacctggcacATCATGTCTTG | |
| | CCTGTATAAAtgagctctagcaatTTTATACAGGCAAGACATGATgccc | |
| | ggcagtgctacaacatcaatgccaaggccgtggggcagctgatggtttg | |
| | ggctcccaacttcccagccaggtgcttctgcag | |
| miR-150 | ggacctgggtataaggcagggactgggcccacggggaggcagcgtcccc | 38 |
| | gaggcagcagcggcagcggcggctcctctccccatggccctgATCATGT | |
| | CTTGCCTGTATAAActgggctcagaccTTTATACAGGCAAGACATGATg | |
| | gacctggggaccccggcaccggcaggccccaaggggtgaggtgagcggg | |
| | cattgggacctcccctccctgtactcccatct | |
| miR-155 | acaaaccaggaaggggaaatctgtggtttaaattctttatgcctcatcc | 39 |
| | tctgagtgctgaaggcttgctgtaggctgtatgctgATCATGTCTTGCC | |
| | TGTATAAAtttgcctccaactgaTTTATACAGGCAAGACATGATgtgta | |
| | tgatgcctgttactagcattcacatggaacaaattgctgccgtgggagg | |
| | atgacaaagaagcatgagtcaccctgctgg | |
| miR-223 | cctttctctctctttccctctagggtcacatctcccaggatgatctcac | 40 |
| | ttccccacagaagctcttggcctggcctcctgcagtgccacgctcATCA | |
| | TGTCTTGCCTGTATAAAggacactccatgtggtagagTTTATACAGGCA | |
| | AGACATGATagtgcggcacatgcttaccagctctaggccagggcagatg | |
| | ggatatgacgaatggactgccagctggatacaaggatgctca | |
| 5′-Stem | GTTGAAGACACACCCTATTTG | 41 |
| Sequence 3 | | |
| 3′-Stem | CAAATAGGGTGTGTCTTCAAC | 42 |
| Sequence 3 | | |
| miR-16 | cctcaaaaatacaaggatctgatcttctgaagaaaatatatttcttttt | 43 |
| | attcatagctcttatgatagcaatgtcagcagtgcctGTTGAAGACACA | |
| | CCCTATTTGttaagattctaaaattatctCAAATAGGGTGTGTCTTCAA | |
| | Cagtaaggttgaccatactctacagttgtgttttaatgtatattaatgt | |
| | tactaatgtgttttcagttttattgatagtcttttcagtatt | |
| miR-142 | acaaggagggctggggggctcttggagcaggagtcaggaggcctgggca | 44 |
| | gcctgaagagtacacgccgacggacagacagacagtgcagtcaccGTTG | |
| | AAGACACACCCTATTTGaacagcactggagggCAAATAGGGTGTGTCTT | |
| | CAACtgagtgtactgtgggcttcggagatcacgccactgctgccgcccg | |
| | ctgcccgccaccatcttcctcggcgctcggggacctcgtgtg | |
| miR-146b | ttactcatcctgggaacgggagacgattcacagaagaaagcatgcaaga | 45 |
| | gcagcgtccaggctgaaagaactttggccacctggcacGTTGAAGACAC | |
| | ACCCTATTTGtgagctctagcaatCAAATAGGGTGTGTCTTCAACgccc | |
| | ggcagtgctacaacatcaatgccaaggccgtggggcagctgatggtttg | |
| | ggctcccaacttcccagccaggtgcttctgcag | |
| miR-150 | ggacctgggtataaggcagggactgggcccacggggaggcagcgtcccc | 46 |
| | gaggcagcagcggcagcggcggctcctctccccatggccctgGTTGAAG | |
| | ACACACCCTATTTGctgggctcagaccCAAATAGGGTGTGTCTTCAACg | |
| | gacctggggaccccggcaccggcaggccccaaggggtgaggtgagcggg | |
| | cattgggacctcccctccctgtactcccatct | |
| miR-155 | acaaaccaggaaggggaaatctgtggtttaaattctttatgcctcatcc | 47 |
| | tctgagtgctgaaggcttgctgtaggctgtatgctgGTTGAAGACACAC | |
| | CCTATTTGtttgcctccaactgaCAAATAGGGTGTGTCTTCAACgtgta | |
| | tgatgcctgttactagcattcacatggaacaaattgctgccgtgggagg | |
| | atgacaaagaagcatgagtcaccctgctgg | |
| miR-223 | cctttctctctctttccctctagggtcacatctcccaggatgatctcac | 48 |
| | ttccccacagaagctcttggcctggcctcctgcagtgccacgctcGTTG | |
| | AAGACACACCCTATTTGggacactccatgtggtagagCAAATAGGGTGT | |
| | GTCTTCAACagtgcggcacatgcttaccagctctaggccagggcagatg | |
| | ggatatgacgaatggactgccagctggatacaaggatgctca | |
| 5′-Stem | CCCGGTTCACTAAGAATCTTT | 49 |
| Sequence 4 | | |
| 3′-Stem | AAAGATTCTTAGTGAACCGGG | 50 |
| Sequence 4 | | |
| miR-16 | cctcaaaaatacaaggatctgatcttctgaagaaaatatatttcttttt | 51 |
| | attcatagctcttatgatagcaatgtcagcagtgcctCCCGGTTCACTA | |
| | AGAATCTTTttaagattctaaaattatctAAAGATTCTTAGTGAACCGG | |
| | Gagtaaggttgaccatactctacagttgtgttttaatgtatattaatgt | |
| | tactaatgtgttttcagttttattgatagtcttttcagtatt | |
| miR-142 | acaaggagggctggggggctcttggagcaggagtcaggaggcctgggca | 52 |
| | gcctgaagagtacacgccgacggacagacagacagtgcagtcaccCCCG | |
| | GTTCACTAAGAATCTTTaacagcactggagggAAAGATTCTTAGTGAAC | |
| | CGGGtgagtgtactgtgggcttcggagatcacgccactgctgccgcccg | |
| | ctgcccgccaccatcttcctcggcgctcggggacctcgtgtg | |
| miR-146b | ttactcatcctgggaacgggagacgattcacagaagaaagcatgcaaga | 53 |
| | gcagcgtccaggctgaaagaactttggccacctggcacCCCGGTTCACT | |
| | AAGAATCTTTtgagctctagcaatAAAGATTCTTAGTGAACCGGGgccc | |
| | ggcagtgctacaacatcaatgccaaggccgtggggcagctgatggtttg | |
| | ggctcccaacttcccagccaggtgcttctgcag | |
| miR-150 | ggacctgggtataaggcagggactgggcccacggggaggcagcgtcccc | 54 |
| | gaggcagcagcggcagcggcggctcctctccccatggccctgCCCGGTT | |
| | CACTAAGAATCTTTctgggctcagaccAAAGATTCTTAGTGAACCGGGg | |
| | gacctggggaccccggcaccggcaggccccaaggggtgaggtgagcggg | |
| | cattgggacctcccctccctgtactcccatct | |
| miR-155 | acaaaccaggaaggggaaatctgtggtttaaattctttatgcctcatcc | 55 |
| | tctgagtgctgaaggcttgctgtaggctgtatgctgCCCGGTTCACTAA | |
| | GAATCTTTtttgcctccaactgaAAAGATTCTTAGTGAACCGGGgtgta | |
| | tgatgcctgttactagcattcacatggaacaaattgctgccgtgggagg | |
| | atgacaaagaagcatgagtcaccctgctgg | |
| miR-223 | cctttctctctctttccctctagggtcacatctcccaggatgatctcac | 56 |
| | ttccccacagaagctcttggcctggcctcctgcagtgccacgctcCCCG | |
| | GTTCACTAAGAATCTTTggacactccatgtggtagagAAAGATTCTTAG | |
| | TGAACCGGGagtgcggcacatgcttaccagctctaggccagggcagatg | |
| | ggatatgacgaatggactgccagctggatacaaggatgctca | |
| 5′-Stem | AGGGCAAAGCATGATTGTATC | 57 |
| Sequence 5 | | |
| 3′-Stem | GATACAATCATGCTTTGCCCT | 58 |
| Sequence 5 | | |
| miR-16 | cctcaaaaatacaaggatctgatcttctgaagaaaatatattttttttatt | 59 |
| | catagctcttatgatagcaatgtcagcagtgcctAGGGCAAAGCATGATTGT | |
| | ATCttaagattctaaaattatctGATACAATCATGCTTTGCCCTagtaaggt | |
| | tgaccatactctacagttgtgttttaatgtatattaatgttactaatgtgtt | |
| | ttcagttttattgatagtcttttcagtatt | |
| miR-142 | acaaggagggctggggggctcttggagcaggagtcaggaggcctgggcagcc | 60 |
| | tgaagagtacacgccgacggacagacagacagtgcagtcaccAGGGCAAAGC | |
| | ATGATTGTATCaacagcactggagggGATACAATCATGCTTTGCCCTtgagt | |
| | gtactgtgggcttcggagatcacgccactgctgccgcccgctgcccgccacc | |
| | atcttcctcggcgctcggggacctcgtgtg | |
| miR-146b | ttactcatcctgggaacgggagacgattcacagaagaaagcatgcaagagca | 61 |
| | gcgtccaggctgaaagaactttggccacctggcacAGGGCAAAGCATGATTG | |
| | TATCtgagctctagcaatGATACAATCATGCTTTGCCCTgcccggcagtgct | |
| | acaacatcaatgccaaggccgtggggcagctgatggtttgggctcccaactt | |
| | cccagccaggtgcttctgcag | |
| miR-150 | ggacctgggtataaggcagggactgggcccacggggaggcagcgtccccgag | 62 |
| | gcagcagcggcagcggcggctcctctccccatggccctgAGGGCAAAGCATG | |
| | ATTGTATCctgggctcagaccGATACAATCATGCTTTGCCCTggacctgggg | |
| | accccggcaccggcaggccccaaggggtgaggtgagcgggcattgggacctc | |
| | ccctccctgtactcccatct | |
| miR-155 | acaaaccaggaaggggaaatctgtggtttaaattctttatgcctcatcctct | 63 |
| | gagtgctgaaggcttgctgtaggctgtatgctgAGGGCAAAGCATGATTGTA | |
| | TCtttgcctccaactgaGATACAATCATGCTTTGCCCTgtgtatgatgcctg | |
| | ttactagcattcacatggaacaaattgctgccgtgggaggatgacaaagaag | |
| | catgagtcaccctgctgg | |
| miR-223 | cctttctctctctttccctctagggtcacatctcccaggatgatctcacttc | 64 |
| | cccacagaagctcttggcctggcctcctgcagtgccacgctcAGGGCAAAGC | |
| | ATGATTGTATCggacactccatgtggtagagGATACAATCATGCTTTGCCCT | |
| | agtgcggcacatgcttaccagctctaggccagggcagatgggatatgacgaa | |
| | tggactgccagctggatacaaggatgctca | |
| 5′-Stem | GCCTCGTAGATTCAGAGCTAT | 65 |
| Sequence 6 | | |
| 3′-Stem | ATAGCTCTGAATCTACGAGGC | 66 |
| Sequence 6 | | |
| miR-16 | cctcaaaaatacaaggatctgatcttctgaagaaaatatatttctttttatt | 67 |
| | catagctcttatgatagcaatgtcagcagtgcctGCCTCGTAGATTCAGAGC | |
| | TATttaagattctaaaattatctATAGCTCTGAATCTACGAGGCagtaaggt | |
| | tgaccatactctacagttgtgttttaatgtatattaatgttactaatgtgtt | |
| | ttcagttttattgatagtcttttcagtatt | |
| miR-142 | acaaggagggctggggggctcttggagcaggagtcaggaggcctgggcagcc | 68 |
| | tgaagagtacacgccgacggacagacagacagtgcagtcaccGCCTCGTAGA | |
| | TTCAGAGCTATaacagcactggagggATAGCTCTGAATCTACGAGGCtgagt | |
| | gtactgtgggcttcggagatcacgccactgctgccgcccgctgcccgccacc | |
| | atcttcctcggcgctcggggacctcgtgtg | |
| miR-146b | ttactcatcctgggaacgggagacgattcacagaagaaagcatgcaagagca | 69 |
| | gcgtccaggctgaaagaactttggccacctggcacGCCTCGTAGATTCAGAG | |
| | CTATtgagctctagcaatATAGCTCTGAATCTACGAGGCgcccggcagtgct | |
| | acaacatcaatgccaaggccgtggggcagctgatggtttgggctcccaactt | |
| | cccagccaggtgcttctgcag | |
| miR-150 | ggacctgggtataaggcagggactgggcccacggggaggcagcgtccccgag | 70 |
| | gcagcagcggcagcggcggctcctctccccatggccctgGCCTCGTAGATTC | |
| | AGAGCTATctgggctcagaccATAGCTCTGAATCTACGAGGCggacctgggg | |
| | accccggcaccggcaggccccaaggggtgaggtgagcgggcattgggacctc | |
| | ccctccctgtactcccatct | |
| miR-155 | acaaaccaggaaggggaaatctgtggtttaaattctttatgcctcatcctct | 71 |
| | gagtgctgaaggcttgctgtaggctgtatgctgGCCTCGTAGATTCAGAGCT | |
| | ATtttgcctccaactgaATAGCTCTGAATCTACGAGGCgtgtatgatgcctg | |
| | ttactagcattcacatggaacaaattgctgccgtgggaggatgacaaagaag | |
| | catgagtcaccctgctgg | |
| miR-223 | cctttctctctctttccctctagggtcacatctcccaggatgatctcacttc | 72 |
| | cccacagaagctcttggcctggcctcctgcagtgccacgctcGCCTCGTAGA | |
| | TTCAGAGCTATggacactccatgtggtagagATAGCTCTGAATCTACGAGGC | |
| | agtgcggcacatgcttaccagctctaggccagggcagatgggatatgacgaa | |
| | tggactgccagctggatacaaggatgctca | |
| 5′-Stem | ATTCCAATTATGATCACTTTC | 73 |
| Sequence 7 | | |
| 3′-Stem | GAAAGTGATCATAATTGGAAT | 74 |
| Sequence 7 | | |
| miR-16 | cctcaaaaatacaaggatctgatcttctgaagaaaatatatttctttttatt | 75 |
| | catagctcttatgatagcaatgtcagcagtgcctATTCCAATTATGATCACT | |
| | TTCttaagattctaaaattatctGAAAGTGATCATAATTGGAATagtaaggt | |
| | tgaccatactctacagttgtgttttaatgtatattaatgttactaatgtgtt | |
| | ttcagttttattgatagtcttttcagtatt | |
| miR-142 | acaaggagggctggggggctcttggagcaggagtcaggaggcctgggcagcc | 76 |
| | tgaagagtacacgccgacggacagacagacagtgcagtcaccATTCCAATTA | |
| | TGATCACTTTCaacagcactggagggGAAAGTGATCATAATTGGAATtgagt | |
| | gtactgtgggcttcggagatcacgccactgctgccgcccgctgcccgccacc | |
| | atcttcctcggcgctcggggacctcgtgtg | |
| miR-146b | ttactcatcctgggaacgggagacgattcacagaagaaagcatgcaagagca | 77 |
| | gcgtccaggctgaaagaactttggccacctggcacATTCCAATTATGATCAC | |
| | TTTCtgagctctagcaatGAAAGTGATCATAATTGGAATgcccggcagtgct | |
| | acaacatcaatgccaaggccgtggggcagctgatggtttgggctcccaactt | |
| | cccagccaggtgcttctgcag | |
| miR-150 | ggacctgggtataaggcagggactgggcccacggggaggcagcgtccccgag | 78 |
| | gcagcagcggcagcggcggctcctctccccatggccctgATTCCAATTATGA | |
| | TCACTTTCctgggctcagaccGAAAGTGATCATAATTGGAATggacctgggg | |
| | accccggcaccggcaggccccaaggggtgaggtgagcgggcattgggacctc | |
| | ccctccctgtactcccatct | |
| miR-155 | acaaaccaggaaggggaaatctgtggtttaaattctttatgcctcatcctct | 79 |
| | gagtgctgaaggcttgctgtaggctgtatgctgATTCCAATTATGATCACTT | |
| | TCtttgcctccaactgaGAAAGTGATCATAATTGGAATgtgtatgatgcctg | |
| | ttactagcattcacatggaacaaattgctgccgtgggaggatgacaaagaag | |
| | catgagtcaccctgctgg | |
| miR-223 | cctttctctctctttccctctagggtcacatctcccaggatgatctcacttc | 80 |
| | cccacagaagctcttggcctggcctcctgcagtgccacgctcATTCCAATTA | |
| | TGATCACTTTCggacactccatgtggtagagGAAAGTGATCATAATTGGAAT | |
| | agtgcggcacatgcttaccagctctaggccagggcagatgggatatgacgaa | |
| | tggactgccagctggatacaaggatgctca | |
| 5′-Stem | GCAGGTGATAGCTGCAAACAT | 81 |
| Sequence 8 | | |
| 3′-Stem | ATGTTTGCAGCTATCACCTGC | 82 |
| Sequence 8 | | |
| miR-16 | cctcaaaaatacaaggatctgatcttctgaagaaaatatatttctttttatt | 83 |
| | catagctcttatgatagcaatgtcagcagtgcctGCAGGTGATAGCTGCAAA | |
| | CATttaagattctaaaattatctATGTTTGCAGCTATCACCTGCagtaaggt | |
| | tgaccatactctacagttgtgttttaatgtatattaatgttactaatgtgtt | |
| | ttcagttttattgatagtcttttcagtatt | |
| miR-142 | acaaggagggctggggggctcttggagcaggagtcaggaggcctgggcagcc | 84 |
| | tgaagagtacacgccgacggacagacagacagtgcagtcaccGCAGGTGATA | |
| | GCTGCAAACATaacagcactggagggATGTTTGCAGCTATCACCTGCtgagt | |
| | gtactgtgggcttcggagatcacgccactgctgccgcccgctgcccgccacc | |
| | atcttcctcggcgctcggggacctcgtgtg | |
| miR-146b | ttactcatcctgggaacgggagacgattcacagaagaaagcatgcaagagca | 85 |
| | gcgtccaggctgaaagaactttggccacctggcacGCAGGTGATAGCTGCAA | |
| | ACATtgagctctagcaatATGTTTGCAGCTATCACCTGCgcccggcagtgct | |
| | acaacatcaatgccaaggccgtggggcagctgatggtttgggctcccaactt | |
| | cccagccaggtgcttctgcag | |
| miR-150 | ggacctgggtataaggcagggactgggcccacggggaggcagcgtccccgag | 86 |
| | gcagcagcggcagcggcggctcctctccccatggccctgGCAGGTGATAGCT | |
| | GCAAACATctgggctcagaccATGTTTGCAGCTATCACCTGCggacctgggg | |
| | accccggcaccggcaggccccaaggggtgaggtgagcgggcattgggacctc | |
| | ccctccctgtactcccatct | |
| miR-155 | acaaaccaggaaggggaaatctgtggtttaaattctttatgcctcatcctct | 87 |
| | gagtgctgaaggcttgctgtaggctgtatgctgGCAGGTGATAGCTGCAAAC | |
| | ATtttgcctccaactgaATGTTTGCAGCTATCACCTGCgtgtatgatgcctg | |
| | ttactagcattcacatggaacaaattgctgccgtgggaggatgacaaagaag | |
| | catgagtcaccctgctgg | |
| miR-223 | cctttctctctctttccctctagggtcacatctcccaggatgatctcacttc | 88 |
| | cccacagaagctcttggcctggcctcctgcagtgccacgctcGCAGGTGATA | |
| | GCTGCAAACATggacactccatgtggtagagATGTTTGCAGCTATCACCTGC | |
| | agtgcggcacatgcttaccagctctaggccagggcagatgggatatgacgaa | |
| | tggactgccagctggatacaaggatgctca | |
| 5′-Stem | CGGATTTCAGTGGACAGTGAT | 89 |
| Sequence 9 | | |
| 3′-Stem | ATCACTGTCCACTGAAATCCG | 90 |
| Sequence 9 | | |
| miR-16 | cctcaaaaatacaaggatctgatcttctgaagaaaatatatttctttttatt | 91 |
| | catagctcttatgatagcaatgtcagcagtgcctCGGATTTCAGTGGACAGT | |
| | GATttaagattctaaaattatctATCACTGTCCACTGAAATCCGagtaaggt | |
| | tgaccatactctacagttgtgttttaatgtatattaatgttactaatgtgtt | |
| | ttcagttttattgatagtcttttcagtatt | |
| miR-142 | acaaggagggctggggggctcttggagcaggagtcaggaggcctgggcagcc | 92 |
| | tgaagagtacacgccgacggacagacagacagtgcagtcaccCGGATTTCAG | |
| | TGGACAGTGATaacagcactggagggATCACTGTCCACTGAAATCCGtgagt | |
| | gtactgtgggcttcggagatcacgccactgctgccgcccgctgcccgccacc | |
| | atcttcctcggcgctcggggacctcgtgtg | |
| miR-146b | ttactcatcctgggaacgggagacgattcacagaagaaagcatgcaagagca | 93 |
| | gcgtccaggctgaaagaactttggccacctggcacCGGATTTCAGTGGACAG | |
| | TGATtgagctctagcaatATCACTGTCCACTGAAATCCGgcccggcagtgct | |
| | acaacatcaatgccaaggccgtggggcagctgatggtttgggctcccaactt | |
| | cccagccaggtgcttctgcag | |
| miR-150 | ggacctgggtataaggcagggactgggcccacggggaggcagcgtccccgag | 94 |
| | gcagcagcggcagcggcggctcctctccccatggccctgCGGATTTCAGTGG | |
| | ACAGTGATctgggctcagaccATCACTGTCCACTGAAATCCGggacctgggg | |
| | accccggcaccggcaggccccaaggggtgaggtgagcgggcattgggacctc | |
| | ccctccctgtactcccatct | |
| miR-155 | acaaaccaggaaggggaaatctgtggtttaaattctttatgcctcatcctct | 95 |
| | gagtgctgaaggcttgctgtaggctgtatgctgCGGATTTCAGTGGACAGTG | |
| | ATtttgcctccaactgaATCACTGTCCACTGAAATCCGgtgtatgatgcctg | |
| | ttactagcattcacatggaacaaattgctgccgtgggaggatgacaaagaag | |
| | catgagtcaccctgctgg | |
| miR-223 | cctttctctctctttccctctagggtcacatctcccaggatgatctcacttc | 96 |
| | cccacagaagctcttggcctggcctcctgcagtgccacgctcCGGATTTCAG | |
| | TGGACAGTGATggacactccatgtggtagagATCACTGTCCACTGAAATCCG | |
| | agtgcggcacatgcttaccagctctaggccagggcagatgggatatgacgaa | |
| | tggactgccagctggatacaaggatgctca | |
| 5′-Stem | GCAGTTCAAAGCTATCTACAA | 97 |
| Sequence 10 | | |
| 3′-Stem | TTGTAGATAGCTTTGAACTGC | 98 |
| Sequence 10 | | |
| miR-16 | cctcaaaaatacaaggatctgatcttctgaagaaaatatatttctttttatt | 99 |
| | catagctcttatgatagcaatgtcagcagtgcctGCAGTTCAAAGCTATCTA | |
| | CAAttaagattctaaaattatctTTGTAGATAGCTTTGAACTGCagtaaggt | |
| | tgaccatactctacagttgtgttttaatgtatattaatgttactaatgtgtt | |
| | ttcagttttattgatagtcttttcagtatt | |
| miR-142 | acaaggagggctggggggctcttggagcaggagtcaggaggcctgggcagcc | 100 |
| | tgaagagtacacgccgacggacagacagacagtgcagtcaccGCAGTTCAAA | |
| | GCTATCTACAAaacagcactggagggTTGTAGATAGCTTTGAACTGCtgagt | |
| | gtactgtgggcttcggagatcacgccactgctgccgcccgctgcccgccacc | |
| | atcttcctcggcgctcggggacctcgtgtg | |
| miR-146b | ttactcatcctgggaacgggagacgattcacagaagaaagcatgcaagagca | 101 |
| | gcgtccaggctgaaagaactttggccacctggcacGCAGTTCAAAGCTATCT | |
| | ACAAtgagctctagcaatTTGTAGATAGCTTTGAACTGCgcccggcagtgct | |
| | acaacatcaatgccaaggccgtggggcagctgatggtttgggctcccaactt | |
| | cccagccaggtgcttctgcag | |
| miR-150 | ggacctgggtataaggcagggactgggcccacggggaggcagcgtccccgag | 102 |
| | gcagcagcggcagcggcggctcctctccccatggccctgGCAGTTCAAAGCT | |
| | ATCTACAActgggctcagaccTTGTAGATAGCTTTGAACTGCggacctgggg | |
| | accccggcaccggcaggccccaaggggtgaggtgagcgggcattgggacctc | |
| | ccctccctgtactcccatct | |
| miR-155 | acaaaccaggaaggggaaatctgtggtttaaattctttatgcctcatcctct | 103 |
| | gagtgctgaaggcttgctgtaggctgtatgctgGCAGTTCAAAGCTATCTAC | |
| | AAtttgcctccaactgaTTGTAGATAGCTTTGAACTGCgtgtatgatgcctg | |
| | ttactagcattcacatggaacaaattgctgccgtgggaggatgacaaagaag | |
| | catgagtcaccctgctgg | |
| miR-223 | cctttctctctctttccctctagggtcacatctcccaggatgatctcacttc | 104 |
| | cccacagaagctcttggcctggcctcctgcagtgccacgctcGCAGTTCAAA | |
| | GCTATCTACAAggacactccatgtggtagagTTGTAGATAGCTTTGAACTGC | |
| | agtgcggcacatgcttaccagctctaggccagggcagatgggatatgacgaa | |
| | tggactgccagctggatacaaggatgctca | |
| 5′-Stem | CGCCTGTAATCCCAGCACTTT | 105 |
| Sequence 11 | | |
| 3′-Stem | AAAGTGCTGGGATTACAGGCG | 106 |
| Sequence 11 | | |
| miR-16 | cctcaaaaatacaaggatctgatcttctgaagaaaatatattttttttatt | 107 |
| | catagctcttatgatagcaatgtcagcagtgcctCGCCTGTAATCCCAGCAC | |
| | TTTttaagattctaaaattatctAAAGTGCTGGGATTACAGGCGagtaaggt | |
| | tgaccatactctacagttgtgttttaatgtatattaatgttactaatgtgtt | |
| | ttcagttttattgatagtcttttcagtatt | |
| miR-142 | acaaggagggctggggggctcttggagcaggagtcaggaggcctgggcagcc | 108 |
| | tgaagagtacacgccgacggacagacagacagtgcagtcaccCGCCTGTAAT | |
| | CCCAGCACTTTaacagcactggagggAAAGTGCTGGGATTACAGGCGtgagt | |
| | gtactgtgggcttcggagatcacgccactgctgccgcccgctgcccgccacc | |
| | atcttcctcggcgctcggggacctcgtgtg | |
| miR-146b | ttactcatcctgggaacgggagacgattcacagaagaaagcatgcaagagca | 109 |
| | gcgtccaggctgaaagaactttggccacctggcacCGCCTGTAATCCCAGCA | |
| | CTTTtgagctctagcaatAAAGTGCTGGGATTACAGGCGgcccggcagtgct | |
| | acaacatcaatgccaaggccgtggggcagctgatggtttgggctcccaactt | |
| | cccagccaggtgcttctgcag | |
| miR-150 | ggacctgggtataaggcagggactgggcccacggggaggcagcgtccccgag | 110 |
| | gcagcagcggcagcggcggctcctctccccatggccctgCGCCTGTAATCCC | |
| | AGCACTTTctgggctcagaccAAAGTGCTGGGATTACAGGCGggacctgggg | |
| | accccggcaccggcaggccccaaggggtgaggtgagcgggcattgggacctc | |
| | ccctccctgtactcccatct | |
| miR-155 | acaaaccaggaaggggaaatctgtggtttaaattctttatgcctcatcctct | 111 |
| | gagtgctgaaggcttgctgtaggctgtatgctgCGCCTGTAATCCCAGCACT | |
| | TTtttgcctccaactgaAAAGTGCTGGGATTACAGGCGgtgtatgatgcctg | |
| | ttactagcattcacatggaacaaattgctgccgtgggaggatgacaaagaag | |
| | catgagtcaccctgctgg | |
| miR-223 | cctttctctctctttccctctagggtcacatctcccaggatgatctcacttc | 112 |
| | cccacagaagctcttggcctggcctcctgcagtgccacgctcCGCCTGTAAT | |
| | CCCAGCACTTTggacactccatgtggtagagAAAGTGCTGGGATTACAGGCG | |
| | agtgcggcacatgcttaccagctctaggccagggcagatgggatatgacgaa | |
| | tggactgccagctggatacaaggatgctca | |
| 5′-Stem | GCCTGTAATCCCAGCACTTTA | 113 |
| Sequence 12 | | |
| 3′-Stem | TAAAGTGCTGGGATTACAGGC | 114 |
| Sequence 12 | | |
| miR-16 | cctcaaaaatacaaggatctgatcttctgaagaaaatatatttctttttatt | 115 |
| | catagctcttatgatagcaatgtcagcagtgcctGCCTGTAATCCCAGCACT | |
| | TTAttaagattctaaaattatctTAAAGTGCTGGGATTACAGGCagtaaggt | |
| | tgaccatactctacagttgtgttttaatgtatattaatgttactaatgtgtt | |
| | ttcagttttattgatagtcttttcagtatt | |
| miR-142 | acaaggagggctggggggctcttggagcaggagtcaggaggcctgggcagcc | 116 |
| | tgaagagtacacgccgacggacagacagacagtgcagtcaccGCCTGTAATC | |
| | CCAGCACTTTAaacagcactggagggTAAAGTGCTGGGATTACAGGCtgagt | |
| | gtactgtgggcttcggagatcacgccactgctgccgcccgctgcccgccacc | |
| | atcttcctcggcgctcggggacctcgtgtg | |
| miR-146b | ttactcatcctgggaacgggagacgattcacagaagaaagcatgcaagagca | 117 |
| | gcgtccaggctgaaagaactttggccacctggcacGCCTGTAATCCCAGCAC | |
| | TTTAtgagctctagcaatTAAAGTGCTGGGATTACAGGCgcccggcagtgct | |
| | acaacatcaatgccaaggccgtggggcagctgatggtttgggctcccaactt | |
| | cccagccaggtgcttctgcag | |
| miR-150 | ggacctgggtataaggcagggactgggcccacggggaggcagcgtccccgag | 118 |
| | gcagcagcggcagcggcggctcctctccccatggccctgGCCTGTAATCCCA | |
| | GCACTTTActgggctcagaccTAAAGTGCTGGGATTACAGGCggacctgggg | |
| | accccggcaccggcaggccccaaggggtgaggtgagcgggcattgggacctc | |
| | ccctccctgtactcccatct | |
| miR-155 | acaaaccaggaaggggaaatctgtggtttaaattctttatgcctcatcctct | 119 |
| | gagtgctgaaggcttgctgtaggctgtatgctgGCCTGTAATCCCAGCACTT | |
| | TAtttgcctccaactgaTAAAGTGCTGGGATTACAGGCgtgtatgatgcctg | |
| | ttactagcattcacatggaacaaattgctgccgtgggaggatgacaaagaag | |
| | catgagtcaccctgctgg | |
| miR-223 | cctttctctctctttccctctagggtcacatctcccaggatgatctcacttc | 120 |
| | cccacagaagctcttggcctggcctcctgcagtgccacgctcGCCTGTAATC | |
| | CCAGCACTTTAggacactccatgtggtagagTAAAGTGCTGGGATTACAGGC | |
| | agtgcggcacatgcttaccagctctaggccagggcagatgggatatgacgaa | |
| | tggactgccagctggatacaaggatgctca | |
| 5′-Stem | GCAGGAGAATCGCTTGAACTT | 121 |
| Sequence 13 | | |
| 3′-Stem | AAGTTCAAGCGATTCTCCTGC | 122 |
| Sequence 13 | | |
| miR-16 | cctcaaaaatacaaggatctgatcttctgaagaaaatatattttttttatt | 123 |
| | catagctcttatgatagcaatgtcagcagtgcctGCAGGAGAATCGCTTGAA | |
| | CTTttaagattctaaaattatctAAGTTCAAGCGATTCTCCTGCagtaaggt | |
| | tgaccatactctacagttgtgttttaatgtatattaatgttactaatgtgtt | |
| | ttcagttttattgatagtcttttcagtatt | |
| miR-142 | acaaggagggctggggggctcttggagcaggagtcaggaggcctgggcagcc | 124 |
| | tgaagagtacacgccgacggacagacagacagtgcagtcaccGCAGGAGAAT | |
| | CGCTTGAACTTaacagcactggagggAAGTTCAAGCGATTCTCCTGCtgagt | |
| | gtactgtgggcttcggagatcacgccactgctgccgcccgctgcccgccacc | |
| | atcttcctcggcgctcggggacctcgtgtg | |
| miR-146b | ttactcatcctgggaacgggagacgattcacagaagaaagcatgcaagagca | 125 |
| | gcgtccaggctgaaagaactttggccacctggcacGCAGGAGAATCGCTTGA | |
| | ACTTtgagctctagcaatAAGTTCAAGCGATTCTCCTGCgcccggcagtgct | |
| | acaacatcaatgccaaggccgtggggcagctgatggtttgggctcccaactt | |
| | cccagccaggtgcttctgcag | |
| miR-150 | ggacctgggtataaggcagggactgggcccacggggaggcagcgtccccgag | 126 |
| | gcagcagcggcagcggcggctcctctccccatggccctgGCAGGAGAATCGC | |
| | TTGAACTTctgggctcagaccAAGTTCAAGCGATTCTCCTGCggacctgggg | |
| | accccggcaccggcaggccccaaggggtgaggtgagcgggcattgggacctc | |
| | ccctccctgtactcccatct | |
| miR-155 | acaaaccaggaaggggaaatctgtggtttaaattctttatgcctcatcctct | 127 |
| | gagtgctgaaggcttgctgtaggctgtatgctgGCAGGAGAATCGCTTGAAC | |
| | TTtttgcctccaactgaAAGTTCAAGCGATTCTCCTGCgtgtatgatgcctg | |
| | ttactagcattcacatggaacaaattgctgccgtgggaggatgacaaagaag | |
| | catgagtcaccctgctgg | |
| miR-223 | cctttctctctctttccctctagggtcacatctcccaggatgatctcacttc | 128 |
| | cccacagaagctcttggcctggcctcctgcagtgccacgctcGCAGGAGAAT | |
| | CGCTTGAACTTggacactccatgtggtagagAAGTTCAAGCGATTCTCCTGC | |
| | agtgcggcacatgcttaccagctctaggccagggcagatgggatatgacgaa | |
| | tggactgccagctggatacaaggatgctca | |
| MSGV1_BLTP3 | tgaaagaccccacctgtaggtttggcaagctagcttaagtaacgccattttg | 129 |
| A-amiR | caaggcatggaaaatacataactgagaatagagaagttcagatcaaggttag | |
| 150_1G4 | gaacagagagacagcagaatatgggccaaacaggatatctgtggtaagcagt | |
| NYESO1TCR | tcctgccccggctcagggccaagaacagatggtccccagatgcggtcccgcc | |
| | ctcagcagtttctagagaaccatcagatgtttccagggtgccccaaggacct | |
| | gaaaatgaccctgtgccttatttgaactaaccaatcagttcgcttctcgctt | |
| | ctgttcgcgcgcttctgctccccgagctcaataaaagagcccacaacccctc | |
| | actcggcgcgccagtcctccgatagactgcgtcgcccgggtacccgtattcc | |
| | caataaagcctcttgctgtttgcatccgaatcgtggactcgctgatccttgg | |
| | gagggtctcctcagattgattgactgcccacctcgggggtctttcatttgga | |
| | ggttccaccgagatttggagacccctgcctagggaccaccgacccccccgcc | |
| | gggaggtaagctggccagcggtcgtttcgtgtctgtctctgtctttgtgcgt | |
| | gtttgtgccggcatctaatgtttgcgcctgcgtctgtactagttagctaact | |
| | agctctgtatctggcggacccgtggtggaactgacgagttcggaacacccgg | |
| | ccgcaaccctgggagacgtcccagggacttcgggggccgtttttgtggcccg | |
| | acctgagtccaaaaatcccgatcgttttggactctttggtgcacccccctta | |
| | gaggagggatatgtggttctggtaggagacgagaacctaaaacagttcccgc | |
| | ctccgtctgaatttttgctttcggtttgggaccgaagccgcgccgcgcgtct | |
| | tgtctgctgcagcatcgttctgtgttgtctctgtctgactgtgtttctgtat | |
| | ttgtctgagaatatgggcccgggctagcctgttaccactcccttaagtttga | |
| | ccttaggtcactggaaagatgtcgagcggatcgctcacaaccagtcggtaga | |
| | tgtcaagaagagacgttgggttaccttctgctctgcagaatggccaaccttt | |
| | aacgtcggatggccgcgagacggcacctttaaccgagacctcatcacccagg | |
| | ttaagatcaaggtcttttcacctggcccgcatggacacccagaccaggtccc | |
| | ctacatcgtgacctgggaagccttggcttttgacccccctccctgggtcaag | |
| | ccctttGTACAggacctgggtataaggcagggactgggcccacggggaggca | |
| | gcgtccccgaggcagcagcggcagcggcggctcctctccccatggccctgAC | |
| | CATCAGCTGAAGTACTTAActgggctcagaccTTAAGTACTTCAGCTGATGG | |
| | Tggacctggggaccccggcaccggcaggccccaaggggtgaggtgagcgggc | |
| | attgggacctcccctccctgtactcccatctTgtacaccctaagcctccgcc | |
| | tcctcttcctccatccgccccgtctctcccccttgaacctcctcgttcgacc | |
| | ccgcctcgatcctccctttatccagccctcactccttctctaggcgccccca | |
| | tatggccatatgagatcttatatggggcacccccgccccttgtaaacttccc | |
| | tgaccctgacatgacaagagttactaacagcccctctctccaagctcactta | |
| | caggctctctacttagtccagcacgaagtctggagacctctggcggcagcct | |
| | accaagaacaactggaccgaccggtggtacctcacccttaccgagtcggcga | |
| | cacagtgtgggtccgccgacaccagactaagaacctagaacctcgctggaaa | |
| | ggaccttacacagtcctgctgaccacccccaccgccctcaaagtagacggca | |
| | tcgcagcttggatacacgccgcccacgtgaaggctgccgaccccgggggtgg | |
| | accatcctctagaccgcctgataagtcgacGCCACCATGTCTATCGGCCTTC | |
| | TGTGTTGTGCCGCTCTCAGCCTGCTGTGGGCCGGGCCTGTGAACGCCGGCGT | |
| | CACCCAGACTCCGAAGTTCCAGGTCTTGAAAACCGGCCAGTCTATGACCTTG | |
| | CAGTGTGCCCAGGACATGAACCACGAGTACATGAGCTGGTACCGGCAAGACC | |
| | CAGGCATGGGCCTGAGGCTAATCCACTACTCCGTCGGCGCCGGAATCACAGA | |
| | CCAGGGCGAAGTGCCCAACGGCTACAACGTATCTCGCAGCACCACCGAGGAC | |
| | TTCCCCCTGCGCCTGCTGAGCGCCGCTCCAAGCCAGACCAGCGTGTACTTCT | |
| | GCGCCTCCAGCTACGTGGGCAACACAGGGGAGCTGTTCTTCGGCGAGGGTTC | |
| | TCGTCTGACCGTGCTGGAAGACCTGCGGAATGTGACCCCTCCTAAAGTGTCC | |
| | TTGTTTGAGCCATCAAAAGCCGAGATCGCCAACAAGCAGAAGGCCACACTTG | |
| | TTTGTCTGGCCAGAGGCTTTTTTCCCGACCACGTGGAACTGTCTTGGTGGGT | |
| | CAACGGAAAAGAGGTGCACAGCGGCGTGTGCACTGACCCTCAGGCCTACAAG | |
| | GAGAGCAACTACAGCTACTGCTTGTCCAGCAGACTGAGGGTGTCCGCCACTT | |
| | TCTGGCACAACCCTCGCAACCACTTTAGATGCCAGGTGCAGTTCCATGGACT | |
| | GAGCGAGGAAGATAAATGGCCTGAGGGTAGTCCCAAGCCCGTCACCCAGAAC | |
| | ATCAGCGCAGAGGCCTGGGGCAGAGCTGACTGCGGGATCACCAGCGCCTCCT | |
| | ACCAGCAGGGTGTGCTGAGCGCGACCATCCTATACGAGATTCTGCTTGGCAA | |
| | GGCCACGCTGTACGCCGTGCTGGTTTCCACTCTGGTGGTGATGGCTATGGTG | |
| | AAGCGGAAGAACAGCAGGGCCAAGCGCAGTGGTTCGGGAGCCACCAACTTCA | |
| | GCCTCCTGAAGCAGGCAGGTGACGTGGAGGAGAATCCTGGCCCCATGGAAAC | |
| | GCTCCTGGGCCTGCTGATCCTGTGGCTGCAGCTGCAGTGGGTGTCATCCAAG | |
| | CAAGAGGTGACCCAGATCCCTGCCGCCCTGTCAGTGCCAGAGGGAGAGAACC | |
| | TGGTTCTGAACTGCTCTTTTACAGATAGCGCCATTTACAACCTGCAGTGGTT | |
| | CCGCCAGGACCCTGGAAAGGGCCTGACAAGCTTGCTCCTGATCCAAAGCTCC | |
| | CAGCGCGAACAGACAAGCGGCAGACTGAATGCTTCGCTCGACAAATCAAGCG | |
| | GCCGCTCCACACTCTATATCGCGGCTAGCCAGCCGGGCGACTCCGCTACTTA | |
| | CCTGTGTGCCGTGCGGCCTACCTCTGGCGGCTCTTACATCCCGACCTTCGGG | |
| | CGTGGCACCAGTCTGATCGTGCATCCCAATATCCAGAACCCTGAACCTGCTG | |
| | TCTATCAGCTGAAGGACCCCAGATCTCAGGACAGCACCCTGTGCCTGTTCAC | |
| | CGATTTCGACTCCCAAATTAACGTGCCTAAGACCATGGAGAGCGGAACCTTC | |
| | ATTACCGACAAGTGCGTGCTGGATATGAAGGCCATGGATAGCAAGTCCAACG | |
| | GCGCCATCGCTTGGAGCAACCAGACCTCCTTCACCTGTCAGGACATCTTCAA | |
| | GGAGACGAACGCGACCTACCCCTCTTCCGACGTACCCTGCGATGCCACCCTG | |
| | ACCGAGAAGAGCTTCGAGACAGATATGAACCTGAACTTCCAGAATCTGCTGG | |
| | TGATCGTGCTCCGCATCCTGCTTCTGAAGGTGGCCGGCTTCAACCTGCTGAT | |
| | GACACTGAGACTGTGGTCTTCTTGATAAggatccgataaaataaaagatttt | |
| | atttagtctccagaaaaaggggggaatgaaagaccccacctgtaggtttggc | |
| | aagctagcttaagtaacgccattttgcaaggcatggaaaatacataactgag | |
| | aatagagaagttcagatcaaggttaggaacagagagacagcagaatatgggc | |
| | caaacaggatatctgtggtaagcagttcctgccccggctcagggccaagaac | |
| | agatggtccccagatgcggtcccgccctcagcagtttctagagaaccatcag | |
| | atgtttccagggtgccccaaggacctgaaatgaccctgtgccttatttgaac | |
| | taaccaatcagttcgcttctcgcttctgttcgcgcgcttctgctccccgagc | |
| | tcaataaaagagcccacaacccctcactcggcgcgccagtcctccgatagac | |
| | tgcgtcgcccgggtacccgtgtatccaataaaccctcttgcagttgcatccg | |
| | acttgtggtctcgctgttccttgggagggtctcctctgagtgattgactacc | |
| | cgtcagcgggggtctttcatgggtaacagtttcttgaagttggagaacaaca | |
| | ttctgagggtaggagtcgaatattaagtaatcctgactcaattagccactgt | |
| | tttgaatccacatactccaatactcctgaaatccatcgatggagttcattat | |
| | ggacagcgcagaaagagctggggagaattgtgaaattgttatccgctcacaa | |
| | ttccacacaacatacgagccggaagcataaagtgtaaagcctggggtgccta | |
| | atgagtgagctaactcacattaattgcgttgcgctcactgcccgctttccag | |
| | tcgggaaacctgtcgtgccagctgcattaatgaatcggccaacgcgcgggga | |
| | gaggcggtttgcgtattgggcgctcttccgcttcctcgctcactgactcgct | |
| | gcgctcggtcgttcggctgcggcgagcggtatcagctcactcaaaggcggta | |
| | atacggttatccacagaatcaggggataacgcaggaaagaacatgtgagcaa | |
| | aaggccagcaaaaggccaggaaccgtaaaaaggccgcgttgctggcgttttt | |
| | ccataggctccgcccccctgacgagcatcacaaaaatcgacgctcaagtcag | |
| | aggtggcgaaacccgacaggactataaagataccaggcgtttccccctggaa | |
| | gctccctcgtgcgctctcctgttccgaccctgccgcttaccggatacctgtc | |
| | cgcctttctcccttcgggaagcgtggcgctttctcatagctcacgctgtagg | |
| | tatctcagttcggtgtaggtcgttcgctccaagctgggctgtgtgcacgaac | |
| | cccccgttcagcccgaccgctgcgccttatccggtaactatcgtcttgagtc | |
| | caacccggtaagacacgacttatcgccactggcagcagccactggtaacagg | |
| | attagcagagcgaggtatgtaggcggtgctacagagttcttgaagtggtggc | |
| | ctaactacggctacactagaaggacagtatttggtatctgcgctctgctgaa | |
| | gccagttaccttcggaaaaagagttggtagctcttgatccggcaaacaaacc | |
| | accgctggtagcggtggtttttttgtttgcaagcagcagattacgcgcagaa | |
| | aaaaaggatctcaagaagatcctttgatcttttctacggggtctgacgctca | |
| | gtggaacgaaaactcacgttaagggattttggtcatgagattatcaaaaagg | |
| | atcttcacctagatccttttaaattaaaaatgaagttttaaatcaatctaaa | |
| | gtatatatgagtaaacttggtctgacagttaccaatgcttaatcagtgaggc | |
| | acctatctcagcgatctgtctatttcgttcatccatagttgcctgactcccc | |
| | gtcgtgtagataactacgatacgggagggcttaccatctggccccagtgctg | |
| | caatgataccgcgagacccacgctcaccggctccagatttatcagcaataaa | |
| | ccagccagccggaagggccgagcgcagaagtggtcctgcaactttatccgcc | |
| | tccatccagtctattaattgttgccgggaagctagagtaagtagttcgccag | |
| | ttaatagtttgcgcaacgttgttgccattgctacaggcatcgtggtgtcacg | |
| | ctcgtcgtttggtatggcttcattcagctccggttcccaacgatcaaggcga | |
| | gttacatgatcccccatgttgtgcaaaaaagcggttagctccttcggtcctc | |
| | cgatcgttgtcagaagtaagttggccgcagtgttatcactcatggttatggc | |
| | agcactgcataattctcttactgtcatgccatccgtaagatgcttttctgtg | |
| | actggtgagtactcaaccaagtcattctgagaatagtgtatgcggcgaccga | |
| | gttgctcttgcccggcgtcaatacgggataataccgcgccacatagcagaac | |
| | tttaaaagtgctcatcattggaaaacgttcttcggggcgaaaactctcaagg | |
| | atcttaccgctgttgagatccagttcgatgtaacccactcgtgcacccaact | |
| | gatcttcagcatcttttactttcaccagcgtttctgggtgagcaaaaacagg | |
| | aaggcaaaatgccgcaaaaaagggaataagggcgacacggaaatgttgaata | |
| | ctcatactcttcctttttcaatattattgaagcatttatcagggttattgtc | |
| | tcatgagcggatacatatttgaatgtatttagaaaaataaacaaataggggt | |
| | tccgcgcacatttccccgaaaagtgccacctgacgtctaagaaaccattatt | |
| | atcatgacattaacctataaaaataggcgtatcacgaggccctttcgtctcg | |
| | cgcgtttcggtgatgacggtgaaaacctctgacacatgcagctcccggagac | |
| | ggtcacagcttgtctgtaagcggatgccgggagcagacaagcccgtcagggc | |
| | gcgtcagcgggtgttggcgggtgtcggggctggcttaactatgcggcatcag | |
| | agcagattgtactgagagtgcaccatatgcggtgtgaaataccgcacagatg | |
| | cgtaaggagaaaataccgcatcaggcgccattcgccattcaggctgcgcaac | |
| | tgttgggaagggcgatcggtgcgggcctcttcgctattacgccagctggcga | |
| | aagggggatgtgctgcaaggcgattaagttgggtaacgccagggttttccca | |
| | gtcacgacgttgtaaaacgacggccagtgccacgctctcccttatgcgactc | |
| | ctgcattaggaagcagcccagtagtaggttgaggccgttgagcaccgccgcc | |
| | gcaaggaatggtgcatgcaaggagatggcgcccaacagtcccccggccacgg | |
| | ggcctgccaccatacccacgccgaaacaagcgctcatgagcccgaagtggcg | |
| | agcccgatcttccccatcggtgatgtcggcgatataggcgccagcaaccgca | |
| | cctgtggcgccggtgatgccggccacgatgcgtccggcgtagaggcgattta | |
| | aagacaggatatcagtggtccaggctctagttttgactcaacaatatcacca | |
| | gctgaagcctatagagtacgagccatagataaaataaaagattttatttagt | |
| | ctccagaaaaaggggggaa | |

### Example 11

shRNA TRCN0000422652 suppresses BLTP3A

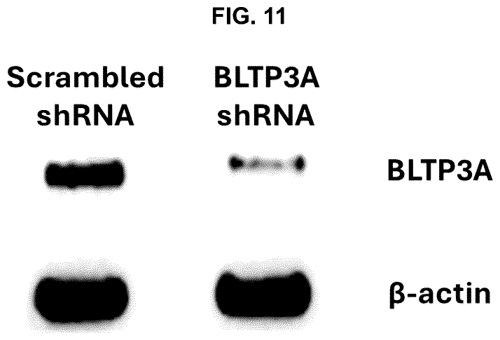

Jurkat cells were transduced to express shRNA TRCN0000422652 (5′-ACCATCAGCTGAAGTACTTAACTCGAGTTAAGTACTTCAGCTGATGGT-3′) (SEQ ID NO: 130) in pMD2G (Addgene; Plasmid No. 12259). BLTP3A levels were assessed after 72 hr by immunoblot (FIG. 11), confirming that the shRNA TRCN0000422652 construct suppressed BLTP3A expression.

What is claimed:

1. A pharmaceutical composition comprising antigen-specific engineered T cells comprising chimeric antigen receptor (CAR)-T cells, T cell receptor (TCR)-T cells, or tumor-infiltrating lymphocytes (TILs) having attenuated or terminated BLTP3A expression.

2. The pharmaceutical composition of claim 1, wherein BLTP3A expression is terminated in the antigen-specific engineered T cells.

3. The pharmaceutical composition of claim 1, wherein the antigen-specific engineered T cells express a nucleic acid molecule specific to BLTP3A that maintains attenuated or terminated BLTP3A expression.

4. The pharmaceutical composition of claim 3, wherein the nucleic acid molecule is a CRISPR-Cas9 construct, a shRNA construct, a siRNA construct, a miRNA construct, or combinations thereof.

5. The pharmaceutical composition of claim 3, wherein the nucleic acid molecule is comprised in a viral vector.

6. The pharmaceutical composition of claim 5, wherein the viral vector comprises a lentiviral vector, a retroviral vector, an adenoviral vector, or an adeno-associated virus (AAV) vector.

7. The pharmaceutical composition of claim 1, wherein the antigen-specific engineered T cells having attenuated or terminated BLTP3A expression have increased anti-cancer effector activity relative to antigen-specific engineered T cells having normal BLTP3A expression.

8. The pharmaceutical composition of claim 1, further comprising one or more pharmaceutically acceptable buffers, salts, carriers, or diluents.

9. A kit comprising:
the pharmaceutical composition of claim 1;
optionally, injection or infusion materials or devices; and
optionally, one or more of packaging, a label, or instructions for use.

10. A method of treating cancer in a subject, the method comprising administering to the subject a therapeutically effective amount of a pharmaceutical composition comprising antigen-specific engineered T cells comprising chimeric antigen receptor (CAR)-T cells, T cell receptor (TCR)-T cells, or tumor-infiltrating lymphocytes (TILs) having attenuated or terminated BLTP3A expression.

11. The method of claim 10, wherein the cancer is a solid-tumor cancer or a liquid cancer.

12. The method of claim 10, wherein the cancer is a solid-tumor cancer selected from pancreatic cancer, breast cancer, lung cancer, brain cancer, neck cancer, ovarian cancer, cervical cancer, prostate cancer, kidney cancer, liver cancer, colorectal cancer, stomach cancer, esophageal cancer, and skin cancer.

13. The method of claim 10, wherein the cancer is ovarian cancer.

14. The method of claim 10, wherein the pharmaceutical composition is administered to the subject by intravenous injection or infusion.

15. The method of claim 10, wherein the pharmaceutical composition delays malignant progression of the cancer in the subject.

16. The method of claim 10, wherein the pharmaceutical composition is cytotoxic to the cancer in the subject.

17. The method of claim 10, further comprising administering to the subject a therapeutically effective amount of one or more additional anti-cancer drugs.

18. The method of claim 17, wherein the one or more additional anti-cancer drugs comprise a PD-1 inhibitor, a PD-L1 inhibitor, a CTLA-4 inhibitor, a TIM-3 inhibitor, a LAG-3 inhibitor, a CD137 agonist, or combinations thereof.

19. A method of producing antigen-specific engineered T cells comprising chimeric antigen receptor (CAR)-T cells, T cell receptor (TCR)-T cells, or tumor-infiltrating lymphocytes (TILs) having attenuated or terminated BLTP3A expression, the method comprising:
treating the antigen-specific engineered T cells with a nucleic acid molecule specific to BLTP3A for a sufficient period of time to attenuate or terminate BLTP3A expression in the antigen-specific engineered T cells.

20. The method of claim 19, wherein treating the antigen-specific engineered T cells with a nucleic acid molecule comprises transfecting the T cells with a CRISPR-Cas9 construct specific to BLTP3A, transducing the T cells with a shRNA construct specific to BLTP3A, transducing the T cells with a siRNA construct specific to BLTP3A, transducing the T cells with a miRNA construct specific to BLTP3A, or combinations thereof.

21. The method of claim 19, wherein the nucleic acid molecule is comprised in a viral vector.

22. The method of claim 21, wherein the viral vector comprises a lentiviral vector, a retroviral vector, an adenoviral vector, or an adeno-associated virus (AAV) vector.

23. The method of claim 19, wherein the nucleic acid molecule is stably expressed in the antigen-specific engineered T cells to maintain attenuated or terminated BLTP3A expression.

24. The method of claim 19, wherein the method terminates BLTP3A expression in the antigen-specific engineered T cells.

25. The method of claim 19, further comprising expanding the antigen-specific engineered T cells.

26. A method of increasing anti-cancer effector activity of antigen-specific engineered T cells comprising chimeric antigen receptor (CAR)-T cells, T cell receptor (TCR)-T cells, or tumor-infiltrating lymphocytes (TILs), the method comprising attenuating or terminating BLTP3A expression in the antigen-specific engineered T cells.

27. The method of claim 26, wherein attenuating or terminating BLTP3A expression in the antigen-specific engineered T cells comprises treating the antigen-specific engineered T cells with a nucleic acid molecule specific to BLTP3A for a sufficient period of time to attenuate or terminate BLTP3A expression in the antigen-specific engineered T cells.

28. The method of claim 27, wherein treating the antigen-specific engineered T cells with a nucleic acid molecule comprises transfecting the T cells with a CRISPR-Cas9 construct specific to BLTP3A, transducing the T cells with a shRNA construct specific to BLTP3A, transducing the T cells with a siRNA construct specific to BLTP3A, transducing the T cells with a miRNA construct specific to BLTP3A, or combinations thereof.
